(19) United States
(12) Patent Application Publication
(10) Pub. No.: US 2021/0179989 A1
(43) Pub. Date: Jun. 17, 2021

(54) SYSTEM AND METHODS FOR OPTOGENETIC EVALUATION OF HUMAN NEUROMUSCULAR FUNCTION

(71) Applicant: The Trustees of Columbia University in the City of New York, New York, NY (US)

(72) Inventors: Olaia Fernandez Vila, San Mateo, CA (US); Gordana Vunjak-Novakovic, New York, NY (US); Stephen Ma, Layton, UT (US); Keith Yeager, Springfield Township, NJ (US)

(21) Appl. No.: 17/118,766

(22) Filed: Dec. 11, 2020

Related U.S. Application Data
(63) Continuation of application No. PCT/US2019/037042, filed on Jun. 13, 2019.
(60) Provisional application No. 62/684,213, filed on Jun. 13, 2018.

(51) Int. Cl. C12M 3/00; A61N 5/06

(52) U.S. Cl. CPC C12M 21/08; A61N 2005/0663; A61N 5/0622

(57) ABSTRACT

A system is provided for evaluating the function of the neuromuscular junction (NMJ) of a subject, which includes a platform including first and second culture chambers separated by a channel; the platform supporting a microtissue culture including: human skeletal myoblasts derived from the subject in the first chamber; a neurosphere derived from the subject, expressing an optogenetic protein in the second chamber, and a hydrogel in the channel to allow axonal sprouting and growth between the myoblasts and neurosphere. A light source is provided for optical stimulation pulses applied to the microtissue culture for activation of the optogenetic protein; and image recordation device for capturing images of the culture in response to the optical stimulation.

### CROSS-REFERENCE TO RELATED APPLICATIONS

This application is a continuation of International Application No. PCT/US2019/037042, entitled “System and Methods For Optogenetic Evaluation of Human Neuromuscular Function” filed Jun. 13, 2019, and which claims priority to U.S. Provisional Application Ser. No. 62/684,213 filed Jun. 13, 2018, entitled “Maturation and Disease of Human Neuromuscular Connectivity Revealed Through Optogenetics,” which is incorporated by reference in its entirety herein.

### GOVERNMENT FUNDING

This invention was made with government support under the grant EB002520 awarded by the National Institutes of Health and the grant W81XWH1810095 awarded by DOD. The government has certain rights in the invention.

### TECHNICAL FIELD

The disclosed subject matter describes a neuromuscular junction (NMJ) tissue-engineered platform. The platform provides a model for the diagnosis and evaluation of disorders of neuromuscular transmission, including myasthenia gravis (MG).

### BACKGROUND

Neuromuscular junctions (NMJs) are the synapses between skeletal fibers and motoneurons, and are disrupted at early stages of various neuromuscular diseases in animal models. For example, the most common disorder of neuromuscular transmission, myasthenia gravis (MG). MG is an autoimmune disorder caused by autoantibodies against the nicotinic acetylcholine receptors, leading to muscular weakness mediated by a decrease in NMJ function.

MG diagnosis is routinely performed based on symptomatology, blood tests for specific antibodies and electrodiagnostic tests. However, the antibody titers typically correlate poorly with disease severity. Furthermore, since not every antibody involved in MG has been identified, some seronegative patients present with the symptoms of MG without testing positive for any identified antibodies. Electrodiagnosis, on the other hand, is an invasive and painful technique and the results can be confused with other pathologies, such as Lambert-Eaton myasthenic syndrome (LEMS), botulism, or motoneuron disease. Thus, electrodiagnosis cannot be used as a standalone diagnostic tool.

The current inability to characterize the NMJ before the first symptoms of disease has prevented the observation of these pathophysiologic processes in human patients.

The study of human neuromuscular diseases has traditionally been performed in animal models, due to the difficulty of performing studies in human subjects. Despite the unquestioned value of animal models, inter-species differences hamper the translation of these findings to clinical trials. background

Human in vitro models of the NMJ enable controllable studies of NMJ function during physiologic development and pathophysiologic disease, providing the basis for both basic science insights as well as translational studies. While previous studies have reported in vitro NMJ formation by human motoneurons and muscle fibers, such approaches include serious limitations. For example, neuromuscular function was difficult to quantify, because (i) NMJs were randomly formed between cells from different sources, and (ii) NMJ function was evaluated manually, which is time consuming and subjective. Therefore, these models have limited utility for systematic studies of neuromuscular physiology and pathology due to biological inconsistencies and inter-observer variation. What is needed is a novel platform to overcome the limitations in evaluating human NMJ.

### SUMMARY

The present disclosure describes a system is provided evaluating the function of the neuromuscular junction (NMJ) of a subject, which includes a platform including first and second culture chambers separated by a gap portion or channel; the platform supporting a microtissue culture including: human skeletal myoblasts derived from the subject in the first chamber; a neurosphere derived from the subject, photosensitive motoneurons expressing the an optogenetic protein in the second chamber, and a hydrogel in the gap portion to allow axonal growth between the myoblasts and neurosphere. A light source is provided for optical stimulation pulses applied to the microtissue culture for activation of the optogenetic protein; and image recordation device for capturing images of the culture in response to the optical stimulation.

In some embodiments, the optogenetic protein is channelrhodopsin-2 (ChR2). In some embodiments, the light source includes a red 647 nm LED for brightfield illumination and a blue 488 nm LED for activation of the optogenetic protein. In some embodiments, the light source provide a ramp stimulation regimen including pulses delivered at successively higher frequencies. In some embodiments, the pulses provided by the light source each have a duration of 100 milliseconds.

In some embodiments, the microtissue define a length of 4 mm.

In some embodiments, the system further includes an image processor executing software configured to receive a stimulation trace of a plurality of optical stimulation pulses by the light source; receive a series of image frames representative of NMJ motion in response to optical stimulation pulses by the light source; extract motion by subtracting every image frame from a baseline frame; create a trace of contractile activity including a plurality of contractions based on the subtraction; align the trace of contractile activity against the stimulation trace; and determine whether each of the optical stimulation pulses was effective based on the time period between an optical stimulation pulse and a contraction.

In another aspect, the platform for evaluating the function of the neuromuscular junction (NMJ) of a subject comprises a body having a bottom, an open top, and first and second wells separated by a first raised lip having a first height. Each well includes a first culture chamber and a second culture chamber disposed adjacent to the first culture chamber. The first and second culture chambers are separated by a second raised lip having a second height. The first and second pillars extend horizontally from a sidewall of the first culture chamber, and a channel is disposed at the bottom of the platform body extending between the first and second culture chambers. In some embodiments, the first height associated with the first raised lip separating the wells is greater than the second height associated with the second culture chamber. The first culture chamber may be disposed in a first section of the platform body, which section includes a bottom surface that is partially closed and an open top portion. A raised embossment may surround the circumference of the first muscle chamber.

In yet another aspect, a tissue engineered three-dimensional model of the neuromuscular junction (NMJ) of a subject is provided, which includes a platform including first and second culture chambers separated by a gap portion; the platform supporting in the first chamber a microtissue culture including: human skeletal myoblasts derived from the subject; in the second chamber a neurosphere derived from the subject, expressing an optogenetic protein, and a hydrogel in the gap portion to allow axonal growth between the myoblasts and neurosphere;

In some embodiments, the optogenetic protein is channelrhodopsin-2 (ChR2). In some embodiments, the human skeletal myoblasts include muscle-derived hiPSCs transduced with lentiviruses carrying the fusion protein hChR2(H134R)-EYFP. In some embodiments, the microtissue is 4 mm long.

A method of evaluating the function of the neuromuscular junction (NMJ) of a subject is provided including: providing a platform including first and second culture chambers separated by a gap portion; the platform supporting a culture including: in the first chamber human skeletal myoblasts derived from the subject; in the second chamber a neurosphere derived from the subject, expressing an optogenetic protein, a hydrogel in the gap portion to allow axonal sprouting and growth between the myoblasts and neurosphere. The method further includes allowing axonal growth between the myoblasts and the neurosphere to form a tissue-engineered NMJ; providing optical stimulation to the second chamber for activation of the optogenetic protein of the tissue-engineered NMJ; measuring displacement of the tissue-engineered NMJ in response to the optical stimulation; and evaluating the tissue culture by determining displacement of tissue in response to the optical stimulation.

In some embodiments, the evaluating includes providing an image processing including software, which when executed by the image processor, cause the processor to receive a stimulation trace of a plurality of optical stimulation pulses by the light source; receive a series of image frames representative of NMJ motion in response to optical stimulation pulses by the light source; extract motion by subtracting every image frame from a baseline frame; create a trace of contractile activity including a plurality of contractions based on the subtraction; align the trace of contractile activity against the stimulation trace; and determine whether each of the optical stimulation pulses was effective based on the time period between an optical stimulation pulse and a contraction.

In some embodiments, the method further includes determining a ratio of effective pulses to total pulses. In some embodiments, the method further includes exposing the tissue-engineered NMJ tissue to serum derived from a second subject; and determining the presence of a neuromuscular disorder in the second subject based on a reduction in effective pulses following exposure of the NMJ tissue to the serum.

In some embodiments, providing optical stimulation includes providing a red 647 nm LED for brightfield illumination and a blue 488 nm LED for activation of the optogenetic protein. In some embodiments, optical stimulation includes providing a ramp stimulation regimen including pulses delivered at successively higher frequencies. In some embodiments, providing optical stimulation includes providing a plurality of pulse each having a duration of 100 milliseconds.

### BRIEF DESCRIPTION OF THE DRAWINGS

A detailed description of various aspects, features and embodiments of the subject matter described herein is provide with reference to the accompanying drawings, which are briefly described below. The drawings are illustrative and are not necessarily drawn to scale, with some components being exaggerated for clarity. The drawings illustrate various aspects and features of the present subject matter and may illustrate one or more embodiment(s) or example(s) of the present subject matter in whole or in part. Together with the description, the drawings serve to explain the principles of the disclosed subject matter.

FIG. 1 is a top view of a bioreactor for use with the tissue-engineering described herein in accordance with exemplary embodiments.

FIG. 2 is a cross-sectional view of the bioreactor of FIG. 1.

FIG. 3 is a perspective view of the bioreactor of FIG. 1.

FIG. 4 is an enlarged top view of the bioreactor of FIG. 1.

FIG. 5 is an enlarged bottom view of the bioreactor of FIG. 1.

FIGS. 6, 7, and 8 are plots illustrating the expression of the endogenous pluripotency genes NANOG, SSEA4 and TRA-1-60.

FIG. 9 illustrates representative cytogenetic analysis of the skeletal myoblast-derived iPSCs showing normal karyotype (n=40).

FIGS. 10-12 illustrate immunofluorescence analysis of expression of pluripotency markers Nanog, Sox2 and Oct3/4 in a skeletal muscle-derived iPSCs.

FIGS. 13-14 illustrates membrane expression of the channelrhodopsin 2-yellow fluorescent protein (ChR2-YFP) complex in human induced pluripotent stem cells (hiPSCs) and hiPSC-dervied motoneurons.

FIG. 15 is an expression of the motoneuronal marker HB9.

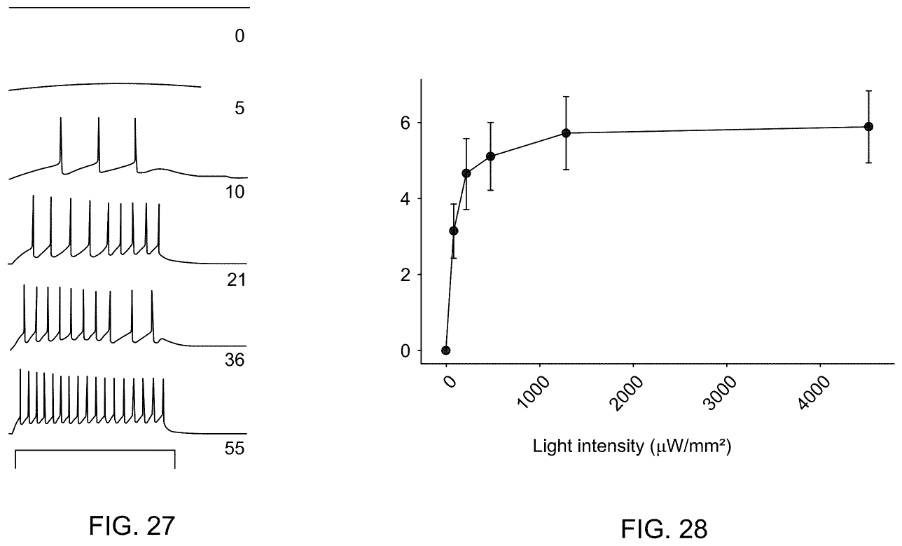

FIGS. 16-18 illustrate immunofluorescence analysis of expression of pluripotency markers NANOG, Sox2 and Oct3/4 in skeletal muscle-derived iPSCs after introduction of the channelrhodopsin-2 gene (ChR2-YFP).

FIG. 19 is a fluorescent image showing neurite extension from the optogenetic motoneuron neurosphere to the skeletal muscle.

FIGS. 20-24 illustrate the evolution of axonal growth from the neurosphere to the muscle tissue during the first week in co-culture.

FIGS. 25-28 illustrate light- and current-evoked action potentials in ChR2-expressing motor neurons derived from hiPSCs cells.

FIGS. 29-30 illustrate action potentials evoked by different duration light exposure.

FIGS. 31-32 illustrate light-evoked currents in ChR2-expressing iPSCs-derived neurons.

FIGS. 33-34 is a confocal image showing innervation of the skeletal microtissue and muscle striation after 10 and 20 days in co-culture.

FIG. 35 illustrates a system for optical stimulation and video processing.

FIGS. 36-38 illustrate an optical stimulation platform in accordance with an exemplary embodiment of the disclosed subject matter.

FIGS. 39-42 are time plots illustrating the correlation between muscle contraction and light pulses for tissue samples with and without optical stimulation.

FIGS. 43-44 are time plots illustrating contractility traces and light stimulation of representative tissue before and after treatment with neurotoxin, respectively.

FIG. 45 is a quantification of tissue responsiveness to light, represented as the corrected fraction of effective light pulse before and after treatment with neurotoxin.

FIG. 46 is a representation of the reduction of the number of spontaneous contractions after treatment with a neurotoxin.

FIGS. 47-49 are time plots illustrating contractility traces and light stimulation of representative tissue at day 9, day 11 and day 16 respectively.

FIG. 50 is a quantification of tissue responsiveness to light, represented as the corrected fraction of effective light pulse over the first three weeks of motorneuron implantation.

FIG. 51 is a representation of forces generated by the skeletal muscle in response to electrical and optical stimulation.

FIGS. 52, 53 and 54 are time plots illustrating contractility traces and light stimulation of representative tissue before, and after treatment with 20% MG serum and after removal of the serum, respectively.

FIG. 55 illustrates the effect of human sera from healthy donors and MG patients on NMJ function.

FIG. 56 illustrates the quantification of NMJ function before and after incubation with serum from MG patients.

FIG. 57 illustrates the differential effect of sera from three different donor at different concentrations.

FIG. 58 illustrates the effect of sera from MG patients with undetectable levels of know MG antibodies (seronegative patients) in the function of the engineered NMJ

### DETAILED DESCRIPTION OF THE DISCLOSED SUBJECT MATTER

The present disclosure provides devices, systems and methods that incorporate tissue-engineered models of the NMJ and allow for the recapitulation of the human physiology in tightly controlled in vitro settings. In addition, the present disclosure provides for tissue-engineered models to diagnose and evaluate MG.

It is to be understood that both the foregoing general description and the following detailed description are exemplary and explanatory only, and are not restrictive of the invention, as claimed. In this description, the use of the singular includes the plural, the word “a” or “an” means “at least one,” and the use of “or” means “and/or,” unless specifically stated otherwise. Furthermore, the use of the term “including,” as well as other forms, such as “includes” and “included” is not limiting. Also, terms such as “element” or “component” encompass both elements or components comprising one unit and elements or components that comprise more than one unit unless specifically stated otherwise.

As used throughout this description, the follow abbreviations are used: BDNF: brain-derived neurotrophic factor; BTX: α-bungarotoxin; ChR2: channelrhodopsin-2; CNTF: ciliary neurotrophic factor; GDNF: glial cell line-derived neurotrophic factor; iPSCs: induced pluripotent stem cells; LED: light emitting diode; LEMS: Lambert-Eaton myasthenic syndrome; MG: myasthenia gravis; NMJ: neuromuscular junction; PBS: phosphate buffered saline; PDMS: polydimethylsiloxane; SEM: standard error of the mean; YFP: yellow fluorescent protein.

The human patient-specific tissue-engineered model of the NMJ, as described herein combines stem cell technology with tissue engineering, optogenetics, microfabrication and image processing. The combination of custom-made hardware and software allows for repeated, quantitative measurements of NMJ function in a user-independent manner.

This model provides for basic and translational research by characterizing in real time the functional changes during physiological and pathological processes.

This system and methods described herein, are believed useful for the study of neuromuscular diseases and drug screening, allowing for the extraction of quantitative functional data from a human, patient-specific system.

The system described herein includes a microfluidic platform comprising the tissue-engineered NMJ tissue, an optical source for stimulating the tissue, an image recordation device for capturing images of the tissue response to the optical stimulation, and an image processor, which analyzes the resulting images to determine the response of the tissue to optical stimulation.

In contrast to two-dimensional co-cultures, microfluidic technologies enable the creation of compartmentalized, three-dimensional tissues that better reproduce human physiology, with a space between the neurosphere and the skeletal tissue that allows the visualization of axonal sprouting and recession under biomimetic conditions. Furthermore, individual three-dimensional tissues are easily traceable and measurable, allowing for systematic analysis of functional changes in individual motor units.

The incorporation of optogenetic proteins to generate photosensitive motoneurons allows for the specific activation of motoneurons without directly stimulating the skeletal muscle tissue, in contrast with electrical stimulation.

Human induced pluripotent stem cells (hiPSCs) can be induced to differentiate towards a variety of cell lineages belonging to the neuromuscular system, including motor neurons. Furthermore, advances in reprogramming techniques now allow for the generation of hiPSCs from multiple sources, including blood and skeletal muscle. Therefore, it is now possible to generate complete tissues involving more than one cell type derived from a single human donor, not only guarantying a perfect match among all the cells types involved, but also allowing for the recapitulation of specific genetic backgrounds.

A novel platform is disclosed herein that overcomes the current limitations in evaluating neuromuscular function in in vitro human systems by combining cell and tissue engineering with optogenetics, microfabrication, optoelectronics and video processing. The integration with custom-made video processing software allows for precise measurements of muscle response. Furthermore, by deriving motoneurons and skeletal myotubes from the same donor, a fully human and patient-specific model is generated that will allow for the study of human neuromuscular physiology and pathology in an in vitro setting, filling the gap between animal studies and clinical trials. Donor-specific NMJ models hold great potential for the study of genetic diseases and can be generated even when the specific pathologic mutation is not known.

The result of this integration is the first quantifiable high-throughput system for the automated evaluation of patient-specific human NMJ function. By reducing the requirement for manual analysis, the system described herein enables the analysis of large sample sizes, and eliminates variability and bias in the evaluation.

In accordance with this disclosure, light responsive NMJs are established between photosensitive motoneurons expressing the optogenetic protein channelrhodopsin-2 (ChR2) and non-optogenetic skeletal muscle tissue that have been derived from a single donor. The hardware and software used herein provides for concurrent stimulation of the NMJ and measurement of its functional response. Using this system, neurotoxin-induced disruption of NMJ function can be detected, as well as graded functional improvement of neuromuscular connectivity over time. Finally, the system is able to detect the presence of MG autoantibodies by incorporation of patient serum, showing differential responses to sera from different donors.

Exemplary methods for cell culture and differentiation are discussed below.

Primary skeletal muscle cells and myotube differentiation. Human skeletal muscle cells from healthy donors were obtained from Cook Myosite and expanded in Myotonic Growth Medium (Cook Myosite #MK-4444) for a maximum of 6 passages. Myoblast fusion was induced by culturing confluent myoblasts in a series of defined media. In some embodiments, cells were cultured in high-glucose DMEM (ThermoFisher Scientific #11995065) supplemented with 500 μg/ml of bovine serum albumin (Sigma Aldrich #A9576), 10 ng/mL insulin (ThermoFisher Scientific #12585014, 10 ng/ml Epidermal Growth Factor (ThermoFisher Scientific #PHG0311), and 50 μg/ml Gentamicin (ThermoFisher Scientific #15750-060). On day 7 after differentiation, the media was changed to differentiation medium 2, consisting of Neurobasal-A (ThermoFisher Scientific #A13710-01) supplemented with Glutamax (ThermoFisher Scientific #35050-061), G-5 (ThermoFisher Scientific #17503-012), B27 (ThermoFisher Scientific #17504-044), 10 ng/ml glial cell line-derived neurotrophic factor (GDNF, R&D Systems #212-GD-010/CF), 20 ng/ml brain-derived neurotrophic factor (BDNF, R&D Systems #248-BD-025/CF), 50 ng/ml recombinant human sonic hedgehog (Shh, R&D Systems #1845-SH-100), 0.1 μM retinoic acid (Sigma Aldrich #R2625-50), 10 ng/ml insulin growth factor 1(IGF-1, ThermoFisher Scientific #PHG0078), 1 μM cyclic adenosine monophosphate (cAMP, Sigma Aldrich #A9752), 5 ng/mL human ciliary neurotrophic factor (CNTF, Miltenyl Biotec #130-096-336), 20 ng/ml neurothropin-3 (Cell Sciences #CRN500B), 20 ng/ml neurothropin-4 (Cell Sciences #CRN501B), 100 ng/ml vitronectin (Sigma Aldrich #V8379), 4 μg/ml mouse laminin, and 100 ng/ml agrin (R&D Systems #550-AG-100). Two days later, media was changed to differentiation media 2 without G5. At day 11 after differentiation, media was changed to NbActive4 (BrainBits LLC #Nb4-500) supplemented with 50 U/ml penicillin/streptomycin (ThermoFisher Scientific #15070063). Media was replaced every 2 days.

Stem cell culture. Stem cells were maintained in mTeSR™1 (Stemcell Technologies #85850)+1% v/v penicillin/streptomycin (ThermoFisher Scientific #12430-047). Media was changed daily for three to four days between passages. For passaging, standard 6-well tissue culture plates were pre-coated for stem cell culture with 1 mL/well of Matrigel (ThermoFisher Scientific #CB-40230) diluted in DMEM/F12 (ThermoFisher Scientific #11320-033) at a ratio of 1:80. Plates were stored at 4° C. for up to two weeks before culture. Prior to passaging, plates were incubated at room temperature for one to four hours. Stem cells were dissociated by incubation with ReLeSR (Stem Cell Technologies #5872) for five minutes followed by rigorous mechanical shearing with a P1000 pipette. Stem cells were seeded at a ratio of 1:12 in mTeSR™1+2 μM Y-27632 dihydrochloride (Tocris #1254) in 2 mL total volume per well.

Generation of muscle derived-hiPSCs lines. Primary skeletal muscle cells were reprogrammed using CytoTune-iPS 2.0 Sendai Reprogramming Kit (ThermoFisher Scientific #A16517) that contains Sendai viruses for KOS, hc-Myc and hKlf4. Briefly, 1×105 P1 skeletal muscle cells were plated in one well of a 6-well plate one day before infection. Cells were infected the next day at MOI 10:10:6 of for KOS:hc-Myc:hKlf4 viruses and incubated for 24 h. After 5 days, cells were trypsinized and plated on top of irradiated mouse embryonic fibroblasts (Globalstem). HiPSCs colonies started forming 2 weeks after transduction. Colonies were then picked and expanded in mouse embryonic fibroblast feeders for 5 additional passages, before switching to matrigel-coated plates.

Karyotyping. At passage 3, muscle-derived hiPSCs were tested for normal karyotype (Cell Line Genetics). Twenty clones from two different lines (forty in total) were tested showing normal phenotype.

LV production and infection. A transgenic cell line was created by infection of the muscle-derived hiPSCs with the pLenti-EF1a-hChR2(H134R)-EYFPWPRE construct (Addgene #20942). Plasmids were grown in One Shot™ Stb13™ chemically competent E. coli (ThermoFisher Scientific #C737303) cultured in LB broth (ThermoFisher Scientific #10855), and isolated using E.Z.N.A.® Endo-Free Plasmid Maxi Kit (Omega Biotek #D6926-03). Human embryonic kidney cells HEK-293FT (ThermoFisher Scientific #R700-07) grown in DMEM (ThermoFisher Scientific #) supplemented with 2% v/v of fetal bovine serum (FBS) (Atlanta Biological #S11150) and 50 U/ml penicillin/streptomycin were transfected with 32.73 μg of the ChR2-YFP plasmid, 10.91 μg of viral envelope plasmid (pMD2.G Addgene #12259) and 21.82 μg of packaging construct (pCMV AR8.2, Addgene #12263) using polyethyleneimine (Polysciences #23966). After 60 hours, supernatant was filtered through a 0.45 mm low protein-binding Steriflip-HV, (Millipore #SE1M003M00) and the viral particles were precipitated using the Lenti-X Concentrator (Takara #631231). Viruses were added to the hiPSCs one day after passaging. YFP+ cells were selected by fluorescence-activated cell sorting (BD Influx™) and expanded.

Motoneuron differentiation. Motoneurons were derived from ChR2-expressing transgenic hiPSC lines using a protocol adapted form Maury et al., “Combinatorial analysis of developmental cues efficiently converts human pluripotent stem cells into multiple neuronal subtypes,” Nat Biotechnol 2015; 33(1); 89-96. On day 0, 4 million hiPSCs were transferred to petri dishes for suspension culture in 10 ml of N2/B27 medium composed of Neurobasal (ThermoFisher Scientific #21103-049) and Advanced DMEM/F12 (ThermoFisher Scientific #12634-020) in a ratio 1:1 and supplemented with B27, N2 (ThermoFisher Scientific #17502-048), Glutamax (ThermoFisher Scientific #35050061), 0.5 μM ascorbic acid (Sigma Aldrich #49752), 0.1 mM 2-Mercaptoethanol (ThermoFisher Scientific #21985023), and 50 U/ml penicillin/streptomycin. On day 0 N2/B27 was supplemented with 3 μM CHIR99021 (Tocris #4423/10), 0.2 μM LDN193189 (Miltenyl Biotec #130-103-925), 40 μM SB431542 hydrate (Sigma Aldrich #S4317) and 5 μM Y-27632 dihydrochloride. On day 2, neurospheres were isolated using 37 μm reversible strainers (STEMCELL Technologies #27215) and replated in N2/B27 supplemented with 3 μM CHIR99021, 0.2 μM LDN193189, 40 μM SB431542 hydrate, and 0.1 μM retinoic acid. Thereafter, media was replaced for N2/B27 supplemented with 0.5 μM smoothened agonist (SAG, Millipore #566660), 0.2 μM LDN193189, 40 μM SB431542, and 0.1 μM retinoic acid at day 4; 0.5 μM SAG and 0.1 μM retinoic acid at day 7; 10 μM DAPT (R&D Systems #2634/10) at day 9; and 20 ng/ml BDNF and 10 ng/ml GDNF at day 11.

Neurosphere dissociation. Motoneurons were dissociated for electrophysiological measurements and immunostaining. Accordingly, 15 mm rounded glass coverslips were sterilized with 70% ethanol, placed in a 24 multiwell plate and treated with 20 μg/ml of poly-L-ornithine (Sigma Aldrich #P4957-50) for 24 h followed by a second 24 h treatment with 3 μg/ml laminin in phosphate buffered saline (PBS). Neurospheres were washed with PBS and incubated with Neurosphere Dissociation Media (iXCells Biotechnologies #MD-0021) for 10-20 min at 36 C with occasional agitation. Motoneurons were then filtered using a 40 μm cell-strainer to eliminate cell clumps, spun down and resuspended in Neurobasal supplemented with Glutamax, non-essential amino acid solution (ThermoFisher Scientific #11140-050), N2, B27, 10 ng/ml GDNF, 10 ng/ml BDNF, 10 ng/ml IGF-1, 10 ng/ml CNTF, 10 μM ascorbic acid, 25 μM L-Glutamic (Sigma Aldrich #G5889), 25 μM 2-mercaptoethanol, 1 μM retinoic acid, and 1 μM uridine/fluorodeoxyuridine (Sigma Aldrich #U3750 and #F0503).

Electrophysiology. Experiments were carried out on a Nikon Eclipse TE 3500 inverted microscope equipped with a 40×1.30 NA objective. Neurons were identified using DIC. Conventional voltage and current clamp recordings were performed using a Multiclamp 700B amplifier and a Digidata 1550 digital-to-analog converter (Molecular Devices). The external recording solution contained 145 mM NaCl, 5 mM KCl, 10 mM HEPES, 10 mM glucose, 2 mM CaCl2 and 2 mM MgCl2. The pipette solution contained 130 mM CH3KO3S, 10 mM CH3NaO3S, 1 mM CaCl2, 10 mM EGTA, 10 mM HEPES, 5 mM MgATP and 0.5 mM Na2GTP (pH 7.3, 305 mOsm). Experiments were performed at room temperature (21-23° C.). During current clamp recordings, current was injected to hold the cells at around −60 mV. For current-evoked depolarization, a series of 1 s current steps increasing in amplitude were applied. For calculation of the charge transfer (Q) following activation of ChR2, cells were voltage clamped at −60 mV. Recordings were carried out using the same solutions as those for the current clamp recordings with the addition of 300 nM tetrodotoxin. The area under the current trace during light exposure was normalized to cell capacitance. ChR2 activation was achieved using a Lambda LS light source (Sutter) and fluorescence filter cube containing a 482/35 nm excitation filter (Semrock) mounted to the microscope. Light was delivered through the microscope objective lens. Light intensity was controlled by 1, 5, 10 and 25% transmission neutral density filters (Chroma) mounted in a Lambda LS-2 filter wheel (Sutter). Light intensities were measured using a PM100D Photometer (Thorlabs). Quantification was carried out with Igor Pro v. 6.3 (Wavemetrics) and R using custom-written scripts.

Exemplary bioreactor are discussed below.

Design. The design of the bioreactor is based on a few functional requirements. Passive tension of tissue attachment pillars on the order of 1 um/mN stiffness, muscle tissue size of approximately 4 mm, and neurosphere size of approximately 300 □m. The neurosphere chamber can connect to the muscle chamber through a small channel on the bottom surface of the device. The channel sizing is such that the neurosphere cannot pass through, however axons can extend through this channel to reach the target muscle tissue. A glass bottom allows for real time imaging of the tissues. Each tissue may be cultured in a common medium (total volume 0.5 mL), however tissue specific media may be used. In this case, since the tissue wells connect, through a small channel, a small amount of mixing may occur and a gradient from one media type to the other would be expected.

Fabrication. The bioreactor was designed for a single body, multi well casting in an elastomer. Initial trials used Silgard 184, an RTV silicone (room temperature vulcanization), cast into a CNC machined mold in POM (acetal/delrin). The same body and function could also have been achieved through LSR (liquid silicone rubber) injection, or also with a more scalable approach using TPEs/TPUs molded in tool steel. A 10:1 base/curing agent mixture of polydimethylsiloxane (PDMS) (Ellsworth Adhesives) was casted into the mold, degassed, and cured at 80° C. for 4 hours, after which the devices were peeled off the molds and cut. The devices were then cleaned in an ultrasonic bath (Branson 1800) using 1-hour cycles of soap, isopropanol and distilled water. They were then dried in the oven at 65° C. overnight. The following day the devices were plasma treated (Harrik PDC-32G) and bonded to a glass cover slip previously treated with 1% Pluronic® F-127 (Sigma Aldrich #P2443) for 15 min. Devices were then autoclaved in water and allow to further dry in the oven at 80° C. for several hours.

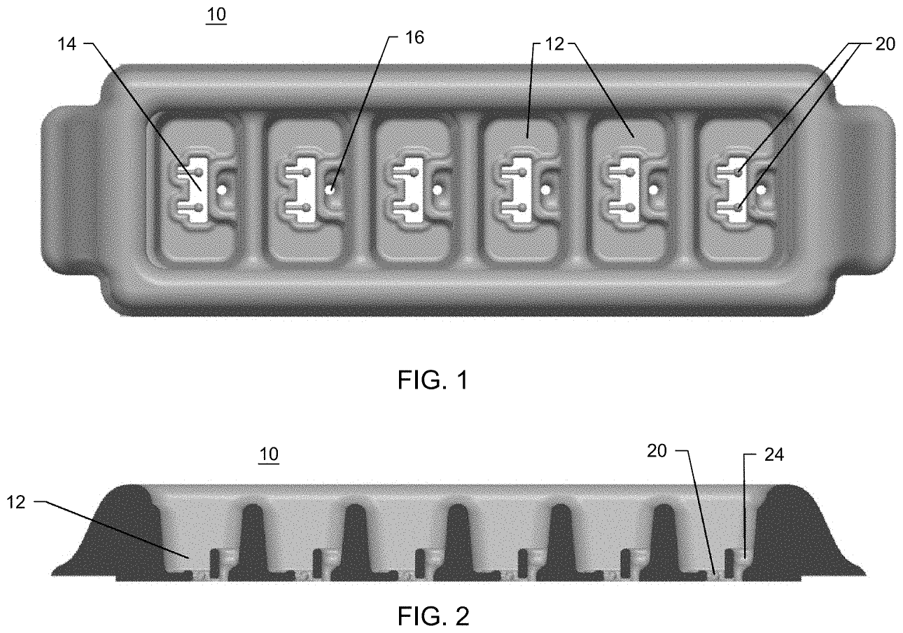

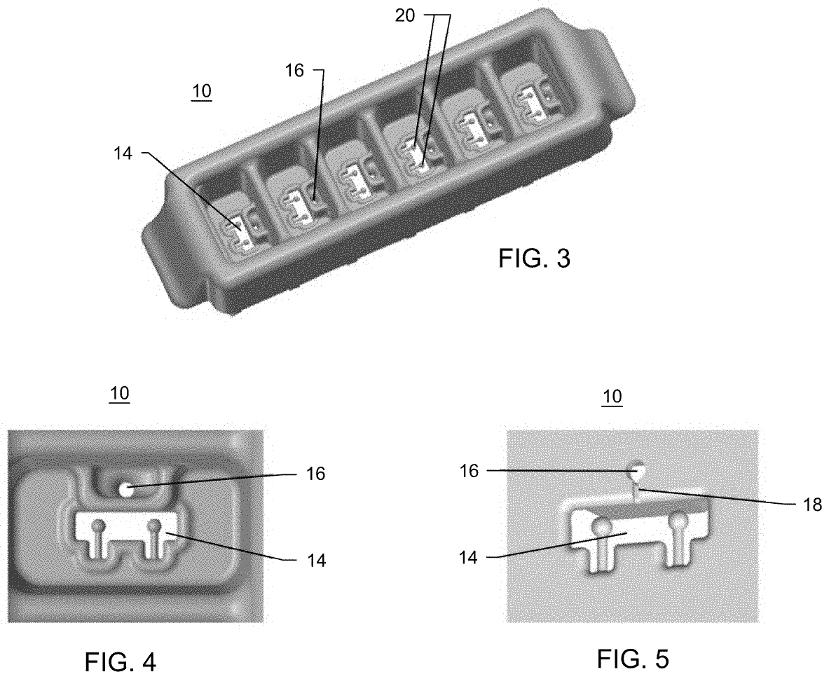

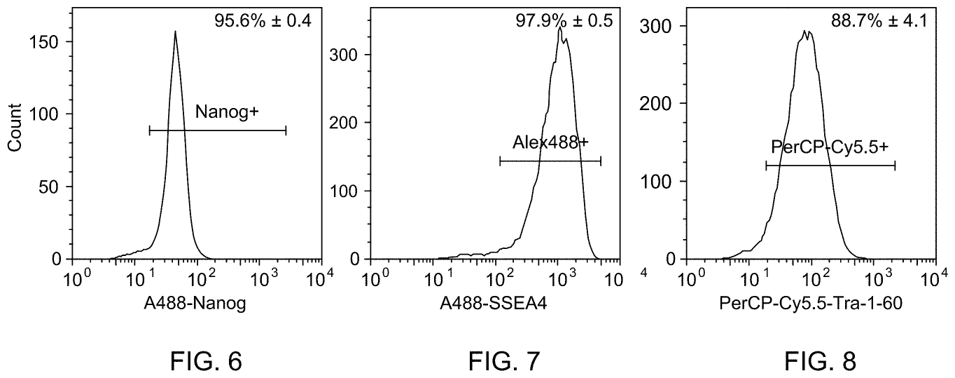

FIGS. 1-5 illustrate an exemplary bioreactor platform device 10 for use with the tissue-engineering described herein. Referring to FIG. 1, platform device 10 includes a body fabricated of PDMS or other suitable material. The body may be elongate having a first end 11, opposing second end 13 and opposing sides 17, 19. As best seen in FIG. 2, the device 10 further includes a bottom 21 and top 23 defining depth or width from top to bottom “W.” Referring back to FIG. 1, a first and second wells (12a-12b) are formed in the platform body 10. AS shown, a plurality of wells may be formed in the platform body. Each well is separated from the adjacent well by a raised lip 27 defining a sidewall.

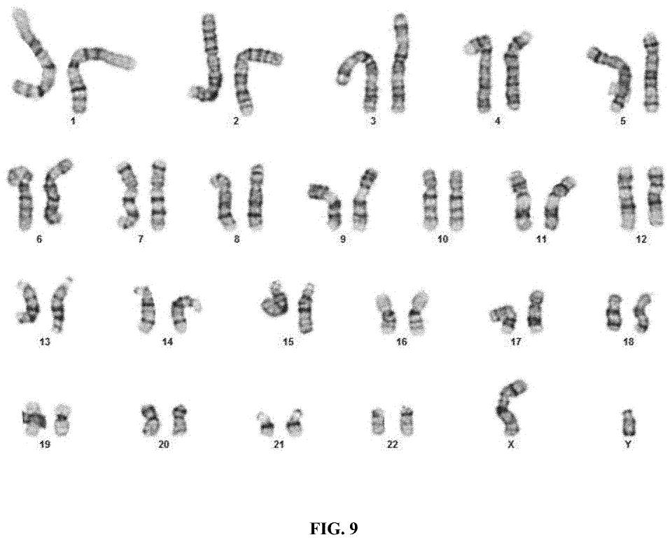

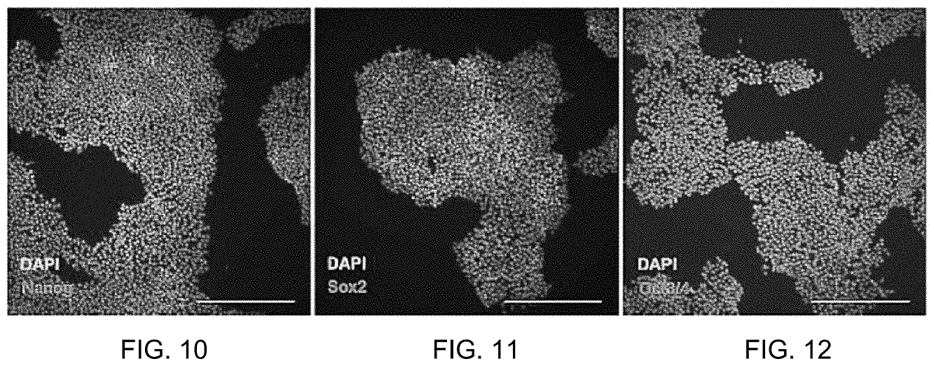

As shown in FIG. 4, each well 12 includes a first section 25 and a second section 29 separated by a raised lip 31 having a height extending from a bottom of well 12. The first section 25 of well 12 includes a first muscle chamber 14. The first muscle chamber defines an aperture through the bottom of well 12. The first muscle chamber includes a raised embossment 33 and a sidewall (best seen in FIG. 5, 35) surrounding the circumference of the first muscle chamber 14. First and second pillars 20a, 20b extend from the sidewall 35 of first muscle chamber 14. The first and second pillars have a horizontal orientation parallel to the bottom of the platform device, and perpendicular to sidewalls of the platform and first muscle chamber 14.

The second section 29 of well 12 further includes a second motoneuron (or neurosphere) chamber 16 defining an aperture through the bottom of well 12. The raised lip 31 separates the second chamber 16 from the first chamber 14. Referring to FIG. 5, a channel (or gap section) 18 is disposed on a bottom surface of the platform device between the first muscle and second motoneuron chambers 16, 14 to allow for axonal growth therebetween. The first muscle chamber 14 may be proximate the second motoneuron chamber. In some embodiments, as shown in FIG. 5, the channel 18 is disposed on the bottom surface of the platform device 10 between the muscle and motorneuron chambers 14, 16. In some embodiments, the distance between the first and second pillars 20a and 20b is 4 mm long to allow larger and stronger tissue formation. Referring to FIGS. 1-5, the first muscle chamber 14 is substantially larger than the second motoneuron chamber 16. In some embodiments the second motorneuron chamber 16 defines a circular aperture, and the first muscle chamber defines a polygonal aperture.

Tissue seeding and culture. Human skeletal myoblasts were seeded at a concentration of 20 million cells/mL in a 4:1 mix of 3 mg/mL collagen I (Corning #354249) and Matrigel. Collagen was diluted in PBS with Phenol Red (Sigma Aldrich #P0290) to achieve the desired concentration, and a 10% solution of NaOH was used to neutralize the gel before adding the Matrigel and resuspend the myoblasts. Then, a 10 μl micropipette was used to fill the muscle chamber 14 with the cell-collagen mixture. After 30 min of polymerization at 37 C, the media reservoirs were filled with Myotonic Growth Media. Myotube differentiation was initiated the day after seeding. Two weeks after myoblast seeding, the muscle chamber 14 and neurosphere chamber 16 and connecting channel 18 were filled with a 4:1 mixture of collagen I 2 mg/mL and Matrigel. HiPSCs-derived neurospheres in the 300-400 μm range were selected using pluriStrainers (PluriSelect #43-50200-03 and #43-50300-03) and seeded in the motoneuron chamber the same hydrogel. Devices were kept in coculture medium, consisting of NbActiv4 supplemented with 10 ng/ml GDNF, 20 ng/ml BDNF, and 50 μM ascorbic acid, from this point, and medium was changed every 2 days.

Optical Stimulation. Measurement of NMJ function was performed with a custom-made optical stimulation platform that uses a 573 nm dichroic mirror (Semrock FF573-Di01-25×36) to couple red (627 nm light emitting diode (LED) (Luxeon Star SP-05-R5) and 594 nm long-pass excitation filter (Semrock BLP01-594R-25)) and blue (470 nm LED (Luxeon Star SP-05-B4) 546 nm short-pass excitation filter (Semrock FF01-546/SP-25)) light sources together. A 594 nm long-pass emission filter distal to the sample (Semrock BLP01-594R-25) was used to filter out blue light for imaging. The LEDs were controlled with an Arduino Uno. For imaging, samples were placed on the stage of an Olympus FSX100 using the red LED from the optical platform as the source of brightfield illumination. The intensity of the 488 nm light used to stimulate the light in this system was 326±8 μW/mm2. The optical stimulation platform was placed on top of the tissue culture plate containing the microfluidic device and aligned so the blue LED was centered on the neurosphere chamber. Movies were acquired using an Andor Zyla 4.2 sCMOS camera through a 10× objective. A ramped stimulation protocol with increasing frequencies (0.2 to 2 Hz in 30 steps) was used to challenge the tissues in terms of number of repetitions and velocity of response in one measurement. Medium was replaced with fresh coculture medium right after optical stimulation.

Electrical Stimulation. Electrical stimulation was performed by placing platinum electrodes (Ladd Research Industries) in both medium reservoirs, connected to an electrical stimulator (Grass s88x). Electrical stimulation was generated by a spatially uniform, pulsatile electrical field (5V intensity, 10 ms in duration, monophasic square waveform) perpendicular to the long axis of the tissue. The parameters were chosen to result in maximum force while avoiding unnecessary electrical tissue damage.

Force calculation. Quantification of the pillar deflection was carried out using the tracking software Tracker (http://physlets.org/tracker). Forces were calculated by multiplying this value by the pillar stiffness.

Contractility analysis. The optical platform includes an image processor for evaluating the images captured by the camera or other image recordation device. For example, data captures includes a stimulation trace, e.g., a time trace of the pulses of optical stimulation, one or more baseline images, and a series of image frames capturing the response of the tissue to stimulation. Brightfield movies were processed to extract motion by subtracting every frame from a baseline frame to get a matrix of differences. The amount of motion at any time point was calculated as the average absolute value of the difference matrix across the frame. This value was calculated for all time points to create the trace of contractile activity, consisting of a series of contractions of the NMJ tissue. This trace was aligned against the stimulation trace by syncing the moment when the red LED was turned on to illuminate the field. Each stimulation pulse was determined as effective if a contraction occurred within 0.1 seconds. The fraction of effective pulses (F) was calculated as the ratio of effective pulses to total light pulses. To account for the possibility that random unstimulated contractions could be correlated with the stimulation pulses by happenstance, an expected fraction of effective pulses (E) was calculated as the expected fraction of pulses to be labeled as effective if the contractions were randomly distributed throughout the time course (total number of contractions×0.1/total time). The fraction of effective pulses was then corrected and normalized to 1 as (F−E)/(1−E).

Bungarotoxin assay. Highly responsive tissues were imaged before and after a 20 min treatment with 5 μg/ml of α-bungarotoxin (BTX) (ThermoFisher Scientific, #B35450). This imaging technique established that contraction of the muscle happens through light-activation of NMJ,

Myasthenia gravis serum treatment. Sera from five myasthenia gravis patients and healthy donors were obtained from Cook Myosite and kept at −80° C. Functional tissues at day 15 after motoneuron seeding were incubated in coculture medium supplemented with 20% serum from either myasthenia gravis patients or healthy donors. NMJ function was measured after 48 h (day 17), and devices were then washed, filled with fresh medium, and imaged again after 48 h (day 19) to measure recovery.

Immunohistochemistry. Cells or tissues were fixed in 4% paraformaldehyde (Santa Cruz #sc-281692) for 20 min at RT, permeabilized with 0.1% Triton X-100 (Sigma Aldrich #T8787) for 15 min at RT, blocked with 10% goat serum (ThermoFisher Scientific #16210072) for 1 hour at RT, incubated with primary antibodies (Table S1) diluted in blocking solution overnight at 4° C., incubated with secondary antibodies (Table S2) for 2 hours at RT and finally, stained with DAPI (#) for 10 min at RT. Cells were rinsed in PBS three times between each step.

TABLE S1
List of primary antibodies.

| | Species and | | | Dilu- |
|---|---|---|---|---|
| Antibody | isotype | Manufacturer | Cat # | tion |
| YPF | Rabbit IgG | Abcam | ab6556 | 1:1000 |
| YPF mAb | Mouse IgG1 | Abcam | ab1218 | 1:1000 |
| α-actinin mAb | Mouse IgG1 | Abcam | ab9465 | 1:100 |
| NANOG mAb | Rabbit IgG | Cell Signaling | D73G4 | 1:200 |
| OCT 3/4 mAb | Rabbit IgG | Cell Signaling | C30A3 | 1:200 |
| SOX2 mAb | Rabbit IgG | Cell Signaling | D6D9 | 1:200 |
| Desmin mAb | Mouse IgG1 | Dako | M076029-2 | 1:100 |
| MyoD | Rabbit IgG | Santa Cruz | sc-760 | 1:100 |
| HB9 mAb | Mouse IgG1κ | DSHB | 81.5C10 | 1:100 |

TABLE S2
List of secondary antibodies.

| | | | | Dilu- |
|---|---|---|---|---|
| Antibody | Conjugation | Manufacturer | Cat # | tion |
| Mouse IgG | AlexaFluor 488 | ThermoFisher | A11001 | 1:1000 |
| | | Scientific | | |
| Rabbit IgG | AlexaFluor 488 | ThermoFisher | A11008 | 1:1000 |
| | | Scientific | | |
| Mouse IgG | Alexa Fluor 555 | ThermoFisher | A21422 | 1:1000 |
| | | Scientific | | |
| Rabbit IgG | Alexa Fluor 568 | ThermoFisher | A11011 | 1:1000 |
| | | Scientific | | |
| Mouse IgG1κ | AlexaFluor 488 | ThermoFisher | A21127 | 1:500 |
| | | Scientific | | |

Flow cytometry. For flow cytometry analysis, hiPSCs were dissociated and incubated with a conjugated antibody for 1 hour at 37 C, 5% CO2. Flow cytometry data was collected on a Bio-Rad S3e™ Cell Sorter. A list of conjugated antibodies can be found in Table S3.

TABLE S3
List of conjugated antibodies for flow cytometry.

| | | | | Dilu- |
|---|---|---|---|---|
| Antibody | Conjugation | Manufacturer | Cat # | tion |
| TRA-1-6 | Cy5.5 | BD Biosciences | 560173 | 1:50 |
| SSEA4 | AlexaFluor 488 | BD Biosciences | 560173 | 1:50 |
| OCT4 | AlexaFluor 488 | BD Biosciences | 560173 | 1:50 |

Statistical analysis. One-way ANOVA analysis was performed to compute F-values for each experiment using MATLAB. Post-hoc Tukey test was used for pairwise comparisons between different groups. A statistical significance threshold of 0.05 was used to determine significance. Values are expressed as mean±SEM.

Results of the experiments discussed herein are described below.

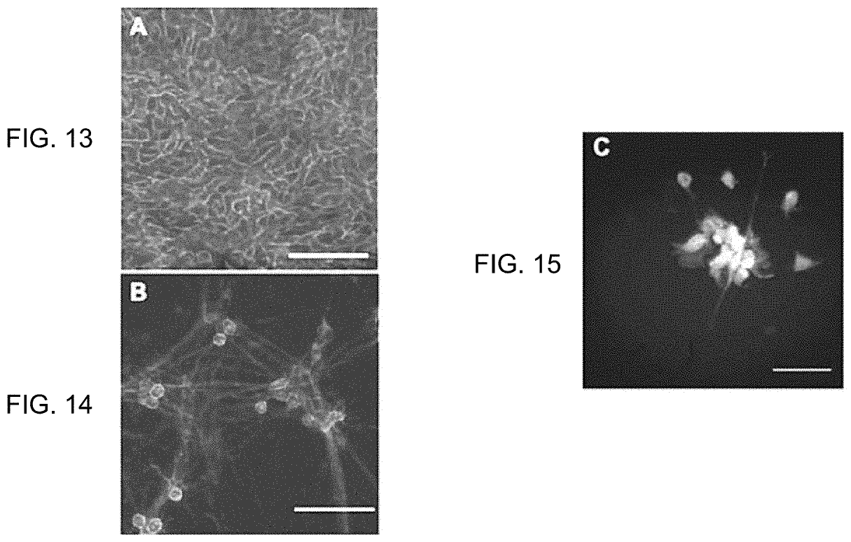

Derivation of skeletal myotubes and optogenetic motoneurons from a single donor. Human primary skeletal myoblasts were reprogrammed into hiPSCs using Sendai viruses containing the pluripotency genes KOS, hc-Myc and hKlf4. Three weeks after the infection, muscle-derived hiPSC colonies expressed the endogenous pluripotency genes NANOG, SSEA4 and TRA-1-60 as shown by flow cytometry in FIGS. 6, 7, 8, respectively.

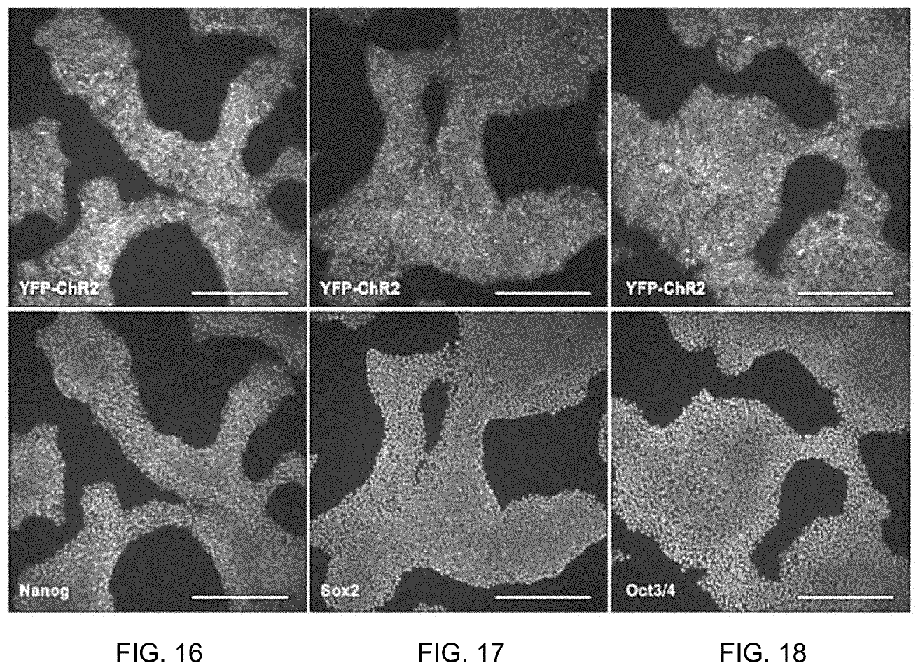

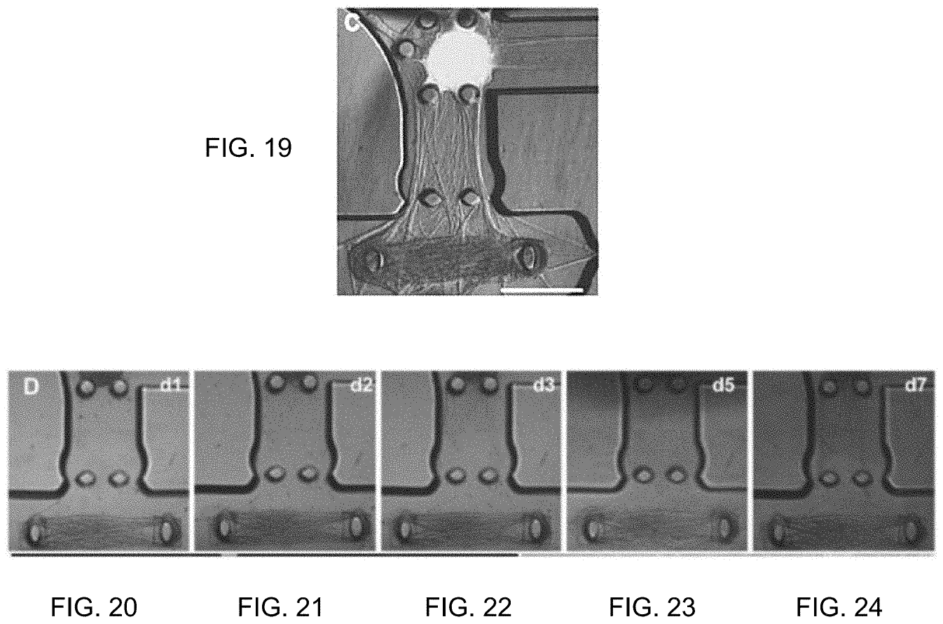

Maintenance of normal karyotype and pluripotency markers NANOG, SOX2 and OCT3/4 was demonstrated after 10 additional passages. FIG. 9 illustrates representative cytogenetic analysis of the skeletal myoblast-derived iPSCs showing normal karyotype (n=40). FIGS. 10, 11, and 12 illustrate immunofluorescence analysis of expression of pluripotency markers Nanog (FIG. 10), Sox2 (FIG. 11) and Oct3/4 (FIG. 12) in a skeletal muscle-derived iPSCs at passage 10. Scale bars: 500 μm.

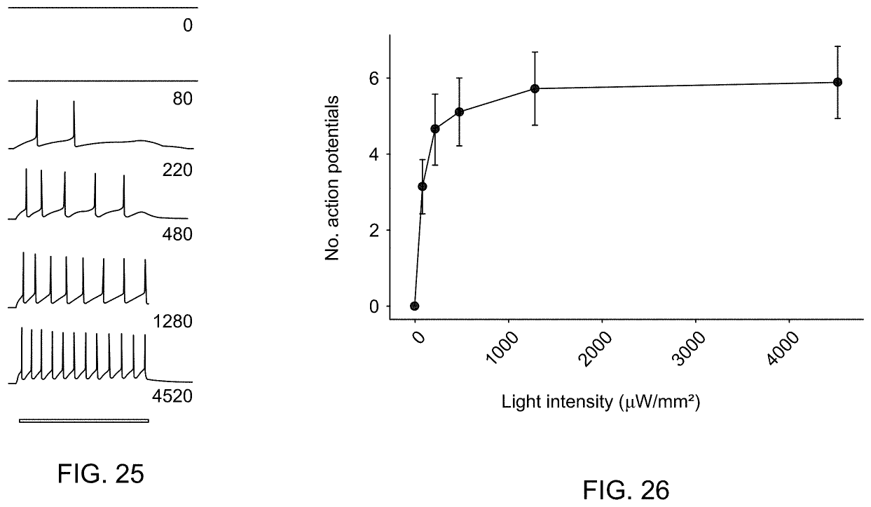

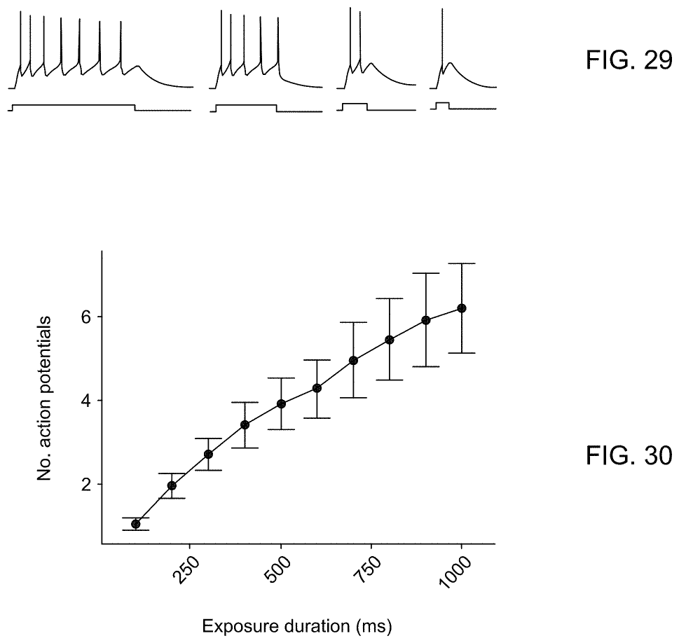

Following expansion, the muscle-derived hiPSCs were transduced with lentiviruses carrying the fusion protein hChR2(H134R)-EYFP. After infection and selection, ChR2-expressing hiPSCs showed transmembrane localization of the construct (FIG. 13) and maintenance of pluripotency markers NANOG (FIG. 16), SOX2 (FIG. 17) and OCT3/4 (FIG. 18.) FIG. 13 illustrates membrane expression of the channelrhodopsin 2-yellow fluorescent protein (ChR2-YFP) complex in human induced pluripotent stem cells (hiPSCs). FIGS. 16, 17, 18 illustrate immunofluorescence analysis of expression of pluripotency markers NANOG, Sox2 and Oct3/4 in skeletal muscle-derived iPSCs after introduction of the channelrhodopsin-2 gene (ChR2-YFP), passage 20. (Scale bars: 50 mm.)

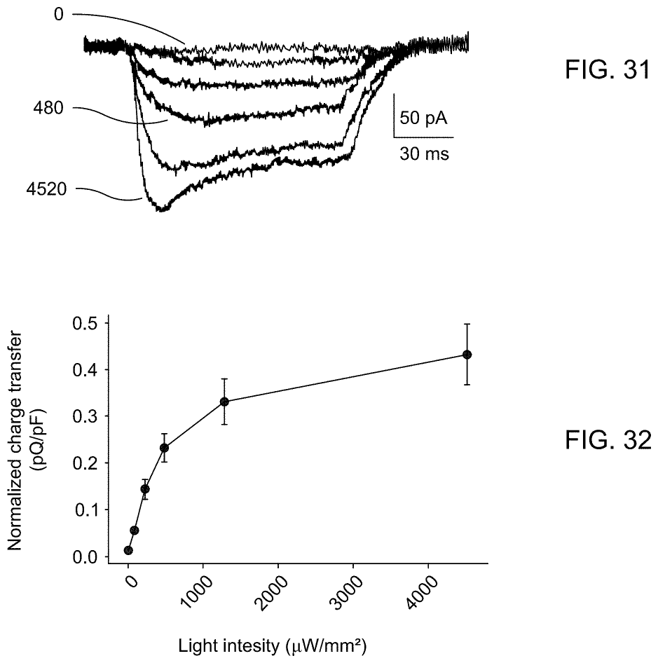

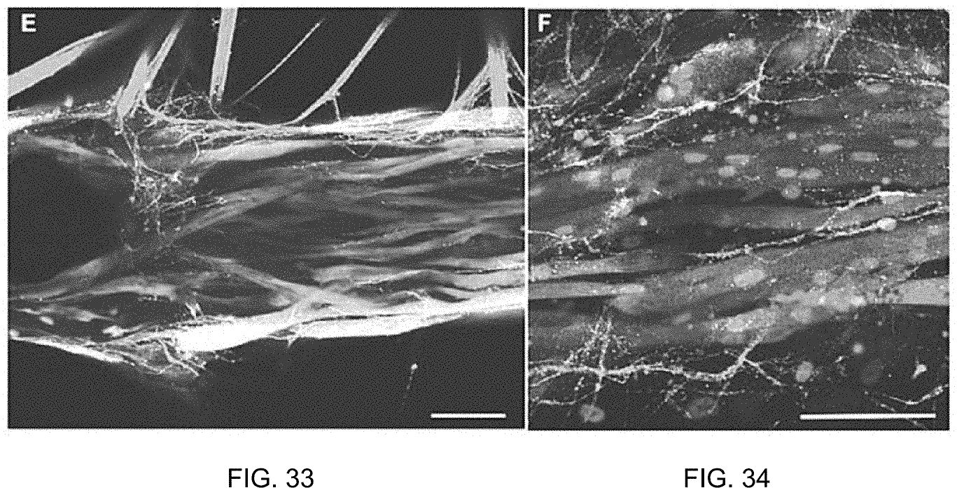

The same cells were used to derive motoneurons that maintained transmembrane localization of the ChR2-eYFP complex (FIG. 14) while also expressing the motoneuron marker HB9 (FIG. 19). FIG. 19 is a fluorescent image showing neurite extension from the optogenetic motoneuron neurosphere to the skeletal muscle. (Scale bar: 500 μm.) Electrophysiological studies of light-evoked action potentials demonstrated the function of ChR2 in a light intensity-dependent manner. FIGS. 20-24 illustrate the evolution of axonal growth from the neurosphere to the muscle tissue during the first week in co-culture (day 1 (FIG. 20); day 2 (FIG. 21); day 3 (FIG. 22); day 5 (FIG. 23); and day 7 (FIG. 24).

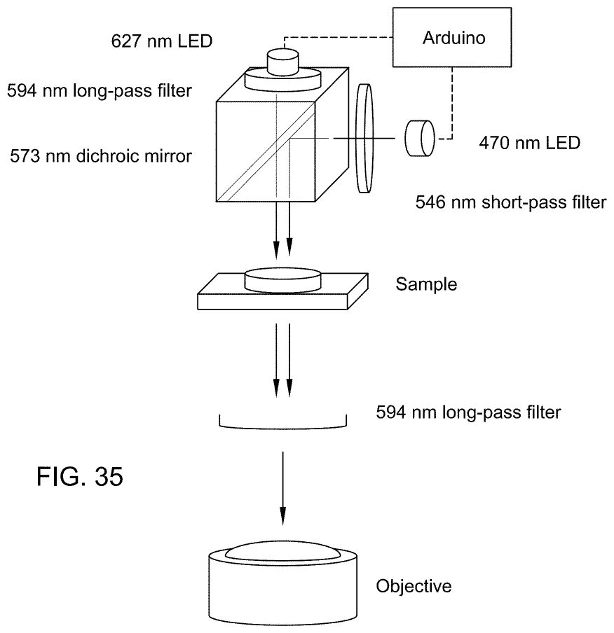

FIGS. 25-28 illustrate light- and current-evoked action potentials in ChR2-expressing motor neurons derived from hiPSCs cells. FIGS. 25-26 illustrate light-evoked action potentials. FIG. 25 illustrates representative membrane potential traces which show action potentials evoked by a 1 s light exposure at different intensities. Light intensities in μW/mm2 are indicated below each trace. FIG. 26 illustrates a graph showing the number of action potentials elicited by a 1 s exposure of light (shown in blue) at various intensities. FIGS. 27-28 illustrate current-evoked action potentials. FIG. 27 illustrates membrane potential traces from the same cell shown in FIGS. 25-26. Action potentials were evoked by a 1 s current injection at incrementally increasing amplitudes. The amplitude of current injection is shown on the right of the trace. Traces are selected which closely match the action potential firing pattern evoked by light. The current injection step period is shown at the base of the column. FIG. 28 illustrates a graph showing the number of action potentials elicited by a 1 s current step of different current injection amplitudes in the same cells shown in FIGS. 25-26. Data points represent the mean number of action potentials and n cells=44 from 4 independent differentiations. (Error bars=SEM.)

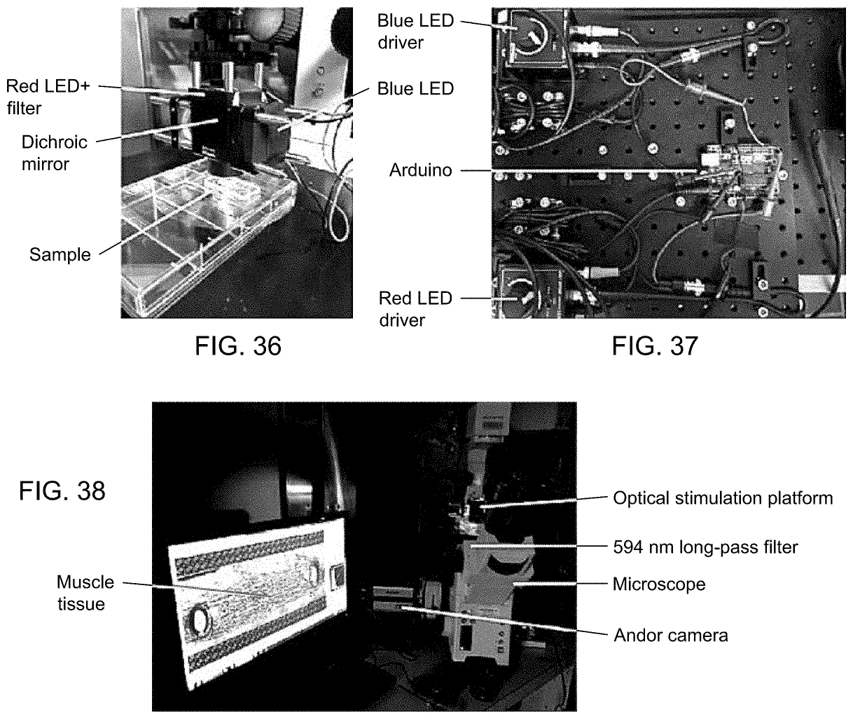

FIGS. 29-30 illustrate action potentials evoked by different duration light exposure. FIG. 29 show representative membrane potential recording from the same cell following exposure of 1000, 500, 200, and 100 ms 219 mW/mm2 light. Bottom trace indicates periods of light exposure. (Scale bar show 20 mV and 200 ms.) FIG. 30 is a plot showing number of action potentials evoked by exposure to different durations of light. n=24 cells from 3 independent differentiations. (Error bars=SEM.)

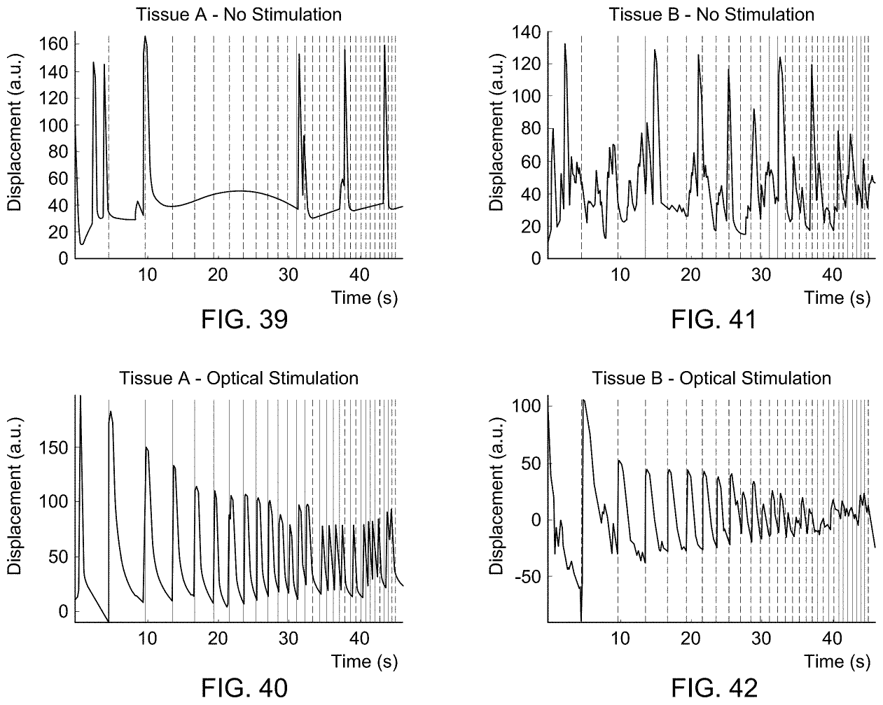

FIGS. 31-32 illustrate light-evoked currents in ChR2-expressing iPSCs-derived neurons. FIG. 31 is a voltage clamp recording showing membrane current traces from a neuron exposed to different intensities of light. For clarity, a subset of traces are labeled with the light intensity in μW/mm2. The period of light exposure is indicated with a bar at the top of the FIG. FIG. 32 is a plot showing the charge transfer normalized to cell capacitance during exposure to 100 ms light at different intensities. Data points represent the mean and SEM. (n cells=14.)

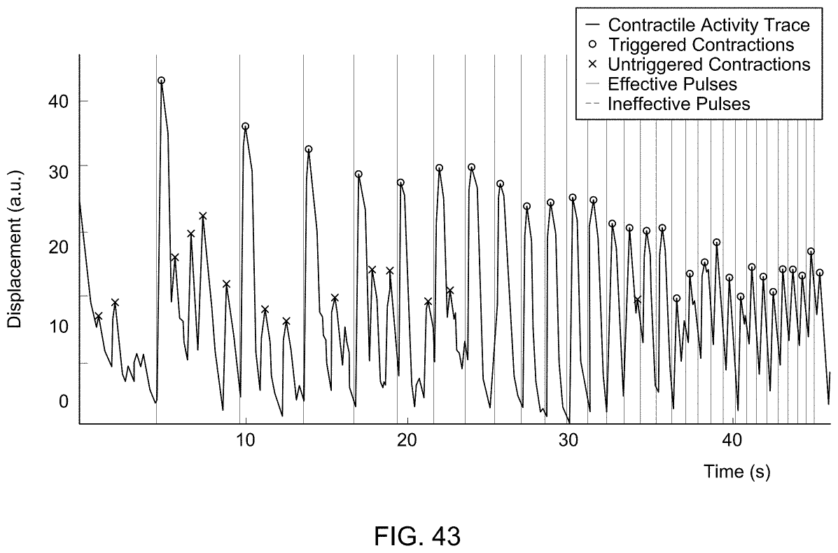

To maintain the same genetic background for both component cells of the NMJ, the myotubes were derived from the original human myoblasts in defined media. Immunostaining of the multinucleated myotubes showed expression of the muscle markers α-actinin, MyoD, Desmin and Myogenin. (FIG. 33).

Determining the right ratio between cell and collagen concentrations was critical for the formation of muscle microtissues. The optimal results were achieved using 20 million cells/ml in 3 mg/ml of collagen and 20% matrigel. Stiffer gels (4 mg/ml) prevented myoblast fusion whereas softer gels (2 mg/ml) resulted in fragile tissues that broke after a few days. Skeletal myoblasts were encapsulated in hydrogel and differentiated into myotubes in the muscle chamber using a series of defined media for 3 weeks. In parallel, ChR2-expressing motoneurons were generated from the muscle-derived hiPSCs in suspension culture. After two weeks, a single motoneuron neurosphere was placed into each motoneuron chamber. Initiation of axonal growth was observed after 24 hours, covering the distance between the neurosphere and the muscle tissue in 5 to 7 days (FIGS. 23, 24). Immunohistology for α-actinin and the YFP-ChR2 complex showed innervation of the muscle microtissues by day 10 of co-culture (FIG. 33) as well as muscle striations at 20 days (FIG. 34).

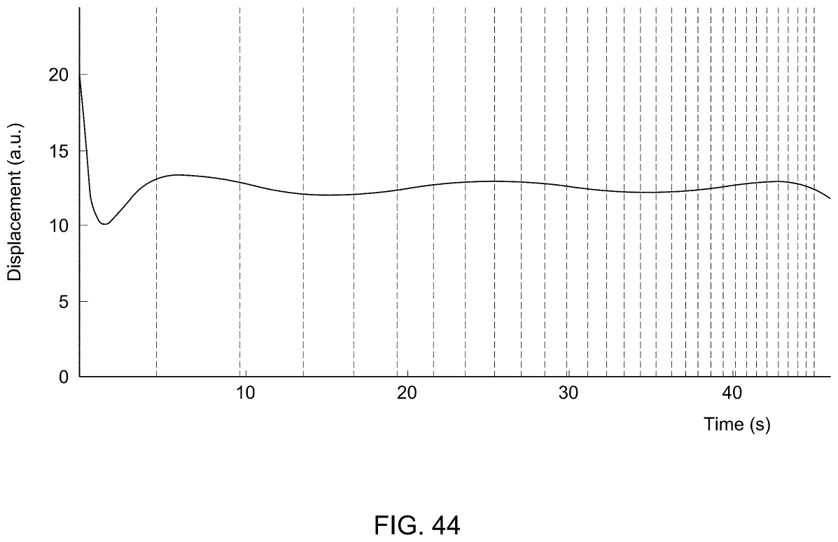

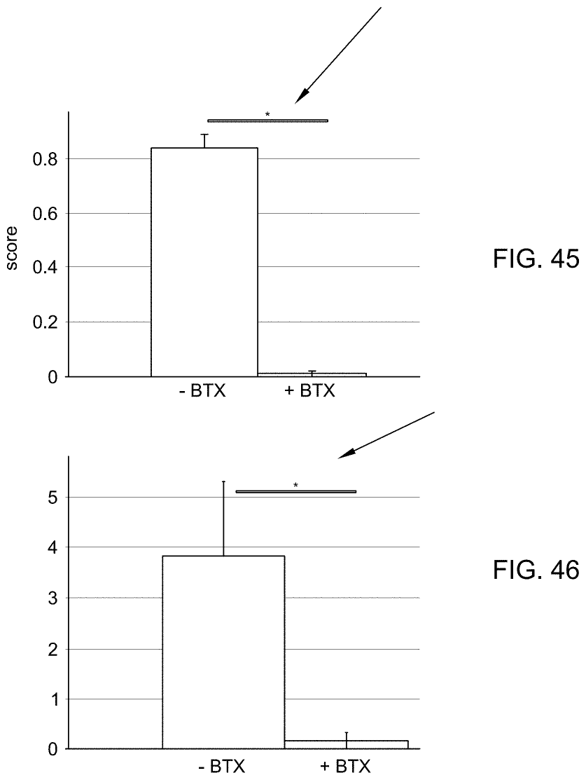

Integration of an optical stimulation platform with custom video processing software for the evaluation of NMJ function. In order to assay NMJ function, an optical stimulation platform with accompanying image-processing code was developed. The optical stimulation platform comprises a 573 nm dichroic mirror to couple red (627 nm LED with a 594 nm long-pass excitation filter) and blue (470 nm LED with a 546 nm short-pass excitation filter) light sources together. The red 627 nm LED is used for brightfield illumination, and a blue 488 nm LED for activation of the ChR2 motoneurons. Blue light was filtered before reaching the objective to prevent photostimulation from interfering with the detection of muscle contractions on brightfield imaging. The LEDs were controlled by an Arduino microprocessor for precise control over the timing of light stimulation, and to allow for its correlation with the imaged muscle contractions using image-processing algorithms (FIG. 35-38). For imaging, samples were placed on the stage of an Olympus FSX100 using the red LED from the optical platform as the source of brightfield illumination. A 594 nm long-pass emission filter is placed on top of the microscope objective to filter out blue light for imaging.

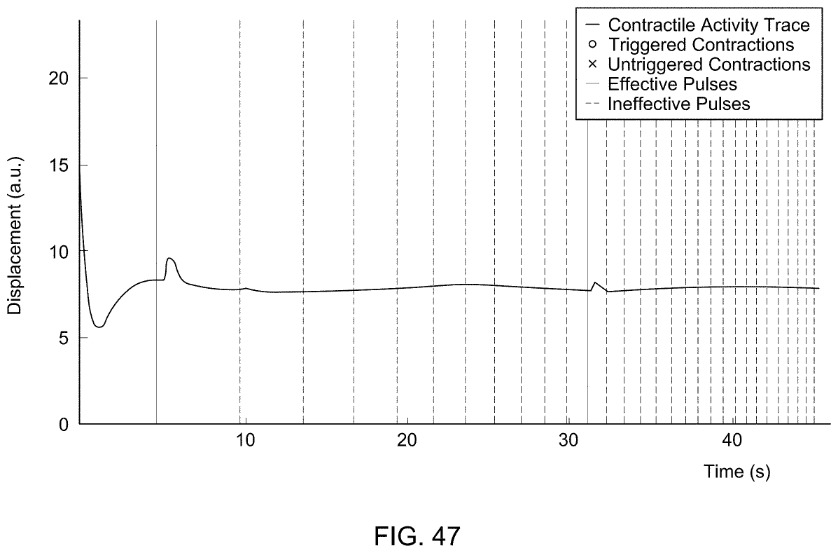

Ramp stimulation regimens consisting of 100-millisecond light pulses delivered at successively higher frequencies were implemented to challenge the tissue in terms of both the number and frequency of repeated contractions. Custom MATLAB code for video processing correlated the light stimulation regimen with the contraction of the muscle tissue (FIGS. 39-42).

Stimulation of muscle tissues with blue light in the absence of motoneurons did not evoke any muscle contractions, proving that muscle contraction in the co-cultures was a result of motoneuron activation and not direct muscle response to light.

Based on the electrophysiological analysis of our motoneurons (FIGS. 29-30) and the power provided by our optical stimulation system (326 μW/mm2) it was expected that 100 ms light pulses would result in the generation of only one action potential. To further test this hypothesis, the tissue response evoked by 1, 10 and 100 ms pulses in the same tissue was compared. The results showed no differences between the response to 10 and 100 ms pulses, whereas short 1 ms pulses are not enough to cause muscle contraction (FIGS. 39-42). FIGS. 39-40 illustrate the response of tissue A. FIGS. 41-42 illustrate the response of tissue 5. No optical stimulation was provided in FIGS. 39 and 41. Optical stimulation was provided in FIGS. 40 and 42. Muscle contractions are shown as a trace, and light pulses are shown as vertical lines. Table C below shows the scoring of the simulated and non-stimulated tissues with and without corrections.

TABLE C

| Tissue | |
|---|---|
| A | B |

| Stimulation | No | Yes | No | Yes |
|---|---|---|---|---|
| Triggered | 2 | 24 | 6 | 36 |
| contractions | | | | |
| Spontaneous | 6 | 2 | 30 | 2 |
| contractions | | | | |
| Fraction of | 0.04 | 0.80 | 0.20 | 0.87 |
| effective pulses | | | | |
| SCORE | 0.02 | 0.77 | 0.005 | 0.84 |

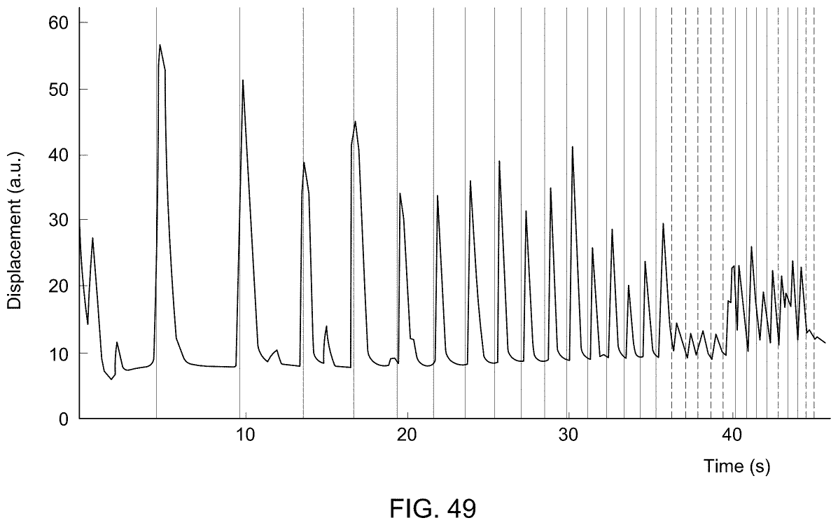

The fraction of effective pulses (the number of light pulses that resulted in muscle contractions) was used as a measure of NMJ function. The probability of contractions randomly happening after a light pulse was calculated based on the total number of contractions during the stimulation time, and used to correct the fraction of effective pulses to obtain a final score (FIGS. 44-45). The algorithm was able to clearly discriminate spontaneous and triggered contractions, as shown in FIG. 44-45 for tissues with different levels of spontaneous activities (Non-stimulated tissues scores=0.02 and 0.005, stimulated scores=0.77 and 0.84 respectively). This strategy allowed evaluation of NMJ function and measure changes in an objective, user independent fashion.

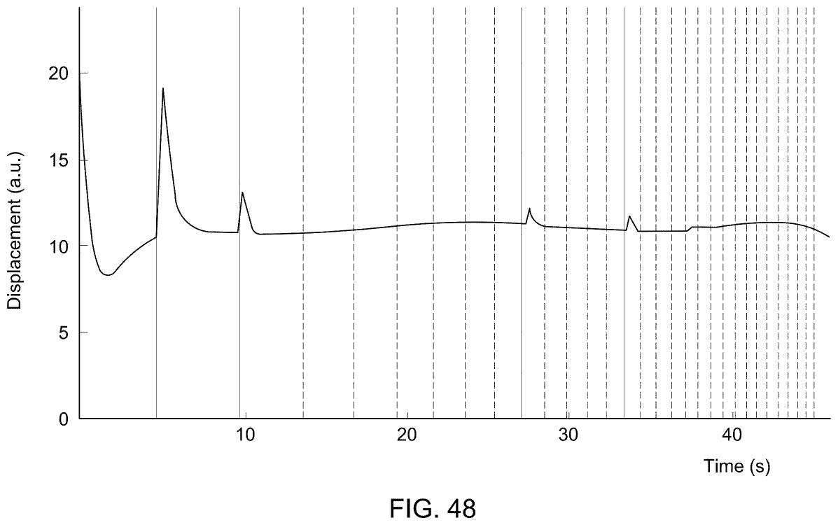

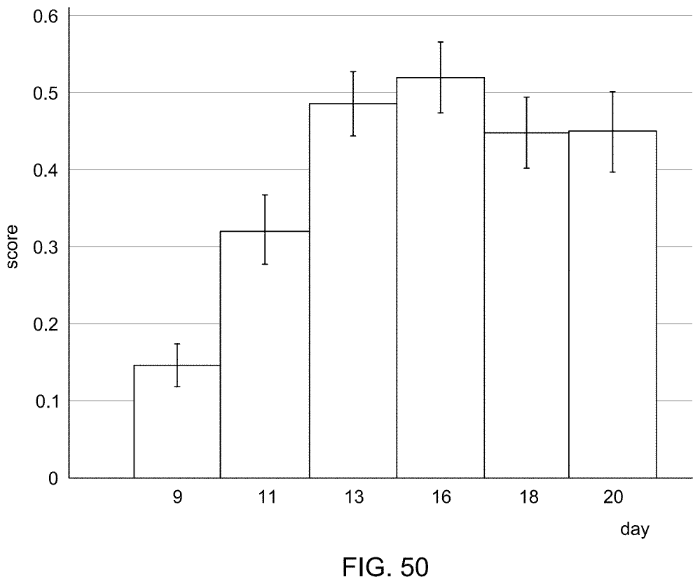

Disruptions of NMJ function due to neurotoxin treatment. With the double purpose of testing our analysis method and to verify that muscle stimulation happens through the NMJs, a set of highly innervated photo-responsive tissues that had been in co-culture for 18 days were selected for analysis using the ramp protocol before (FIG. 43) and after 20 minutes of incubation with 5 μg/ml of the neurotoxin α-bungarotoxin (BTX), that binds specifically and irreversibility to the acetylcholine receptors in the NMJ (FIG. 44). BTX completely stopped both light-triggered and spontaneous contractions, proving that light stimulation of the tissues requires a functional NMJ. Quantification of tissue responsiveness to light before and after BTX treatment showed a complete disruption of NMJ function in all the tissues (average score before treatment=0.84±0.05, after treatment=0.01±0.01, one-way ANOVA F=9·10-8) (FIG. 45) as well as suppression of the spontaneous activity of the muscle tissues (average number of spontaneous contractions before treatment=3.84±1.5, average number of spontaneous contractions after treatment=0.17±0.17, one-way ANOVA F=0.03) (FIG. 46).

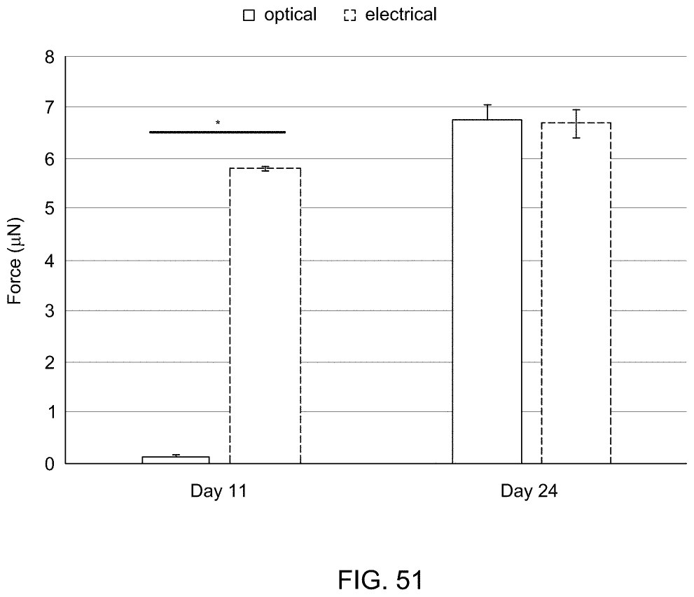

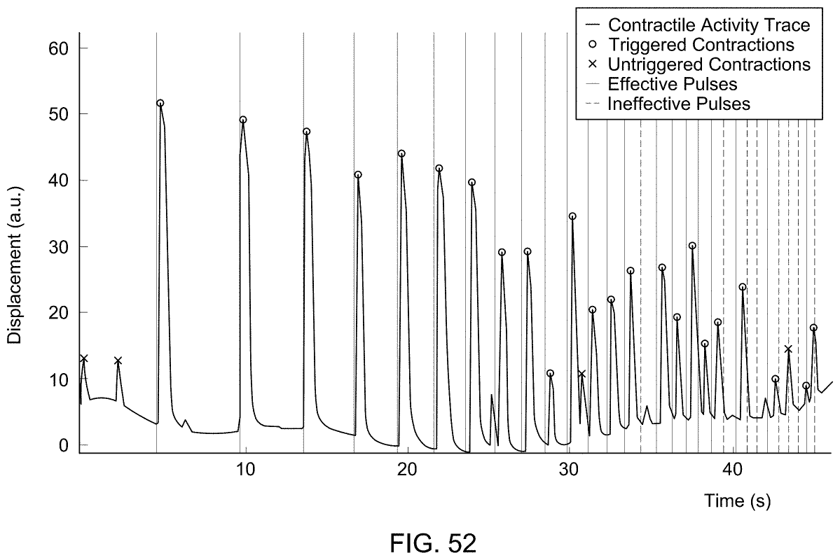

Measurement of physiological changes in NMJ function. To demonstrate the capacity of the system to quantitatively track changes in NMJ function over time, the same group of tissues were imaged for 11 days (day 9 to 20 after motoneuron implantation). Movies and evaluation of a representative tissue at day 9 (FIG. 47, score=0.06), day 13 (FIG. 48, score=0.12) and day 16 (FIG. 49, score=0.71) demonstrate the ability to capture the graded improvements in NMJ function of individual tissues during the physiologic process of innervation. The quantification of movies acquired for 47 tissues in 2 independent experiments documented functional improvement of the neuromuscular synapse during the first week after formation, followed by another week of stable function (average scores: day 9=0.15±0.03, day 11=0.32±0.04, day 16=0.52±0.05) (FIG. 50).

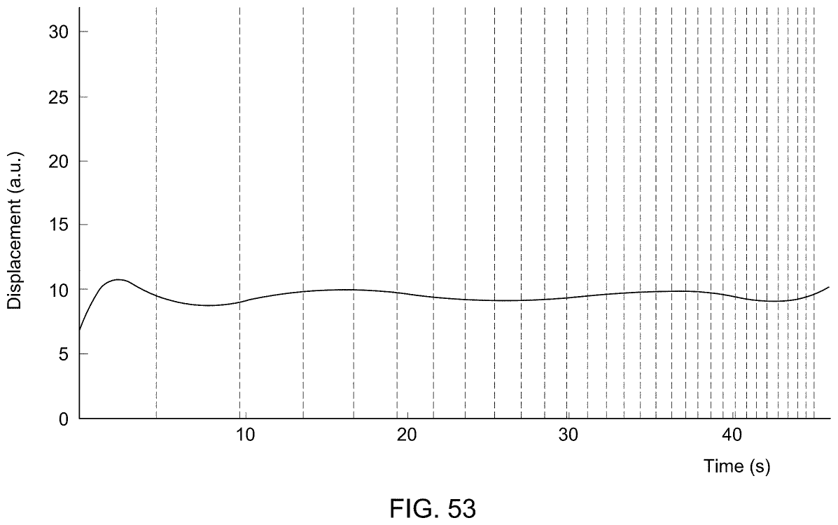

The force generated by the muscle tissues can be calculated as the product of the pillar displacement multiplied by their stiffness. Comparison of the forces generated by the innervated tissues in response to electrical stimulation at early and late stages show no significative differences, whereas light-evoked contractions improve drastically, achieving the same levels as electrically induced forces by day 24 (FIG. 51). This suggests that improvement in light responsiveness of the tissue is not due to increased muscular function but to improvement in the neuromuscular connectivity.

Measurement of pathological changes in NMJ function. To demonstrate the translational utility of our system, the pathological changes in NMJ function caused by MG are recapitulated.

To recapitulate the myasthenic phenotype in the system, we first incorporated pooled sera from 5 patients carrying MG autoantibodies. However, convention systems involved manually opening a fluorescent lamp shutter and recording contractions in randomly chosen fields, thus hindering the reproducibility and decreasing the quantitative power of the system.

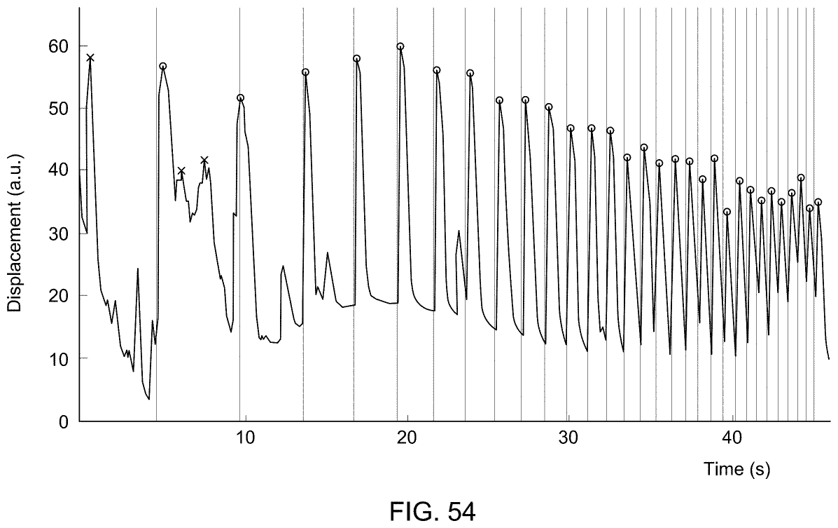

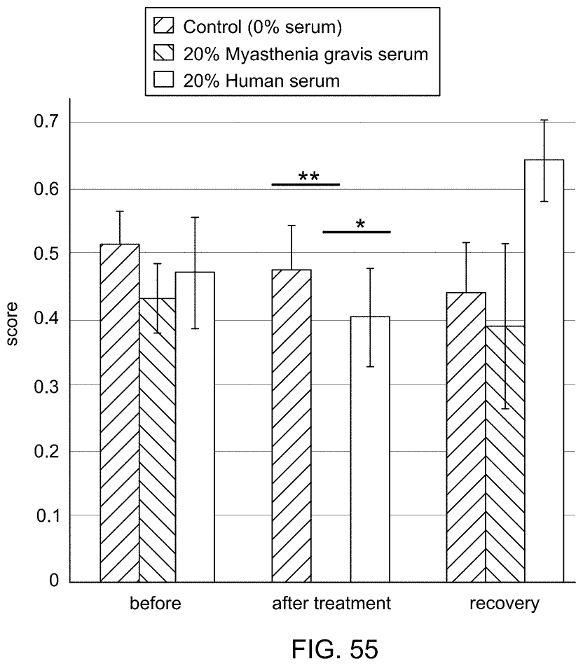

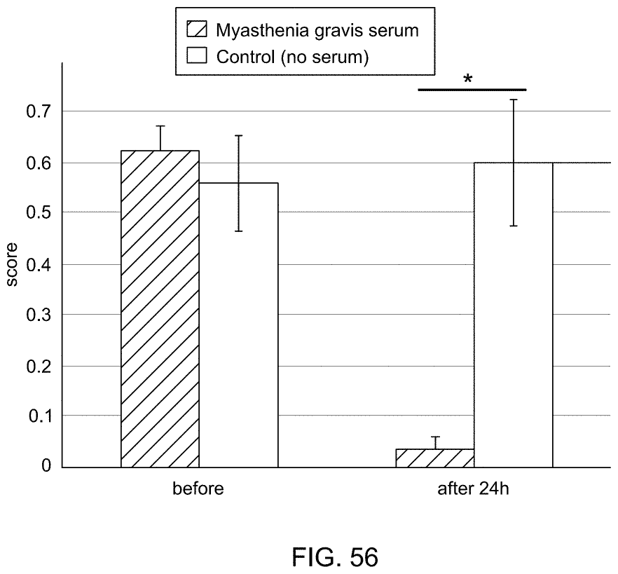

The system described herein allowed for repeated precise measurements of the same tissues before exposure (FIG. 52) and after incubation with MG serum (FIG. 53). After washout of the antibodies (FIG. 54), NMJ function was recovered. Quantification of the results for tissues treated with 20% MG serum for 48 h showed drastically impaired function (before treatment score mean=0.43±0.05; after treatment score mean=10-5±0.00) compared to controls treated with serum from healthy donors (before treatment score mean=0.47±0.07; after treatment score=0.40±0.07, p=0.0015) or non-treated tissues (before treatment score mean=0.51±0.05; after treatment score=0.47±0.07, p=3.5·10-6). MG serum was removed and tissues were washed after imaging. Evaluation of tissue function 48 h after serum removal showed total functional recovery (recovery score mean=0.39±0.11) (FIG. 55). In an independent experiment, a lower dose of MG serum (10%) with a shorter incubation period of 24 h also showed a drastic effect on NMJ function (treated group score=0.04±0.02, control group score=0.60±0.12) (FIG. 56). These results demonstrate the capability of the system to detect and quantify changes in NMJ function, and to model human diseases in vitro.

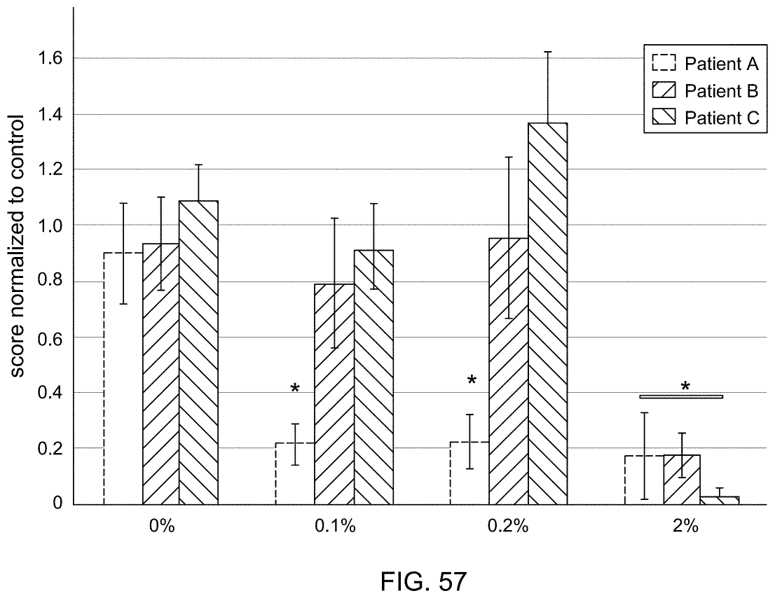

In order to prove the potential of the system as an evaluation tool for myasthenia gravis and other neuromuscular diseases, individual serum from 3 different patients at successively increasing doses was tested. The results show that the system is able to detect changes in the NMJ function at doses as low as 0.1% for one of the patients, whereas it is necessary to increment the serum concentration 20 times to be able to see an effect for the other two patients (FIG. 57). These results indicate that the system is able to detect differential effects from different patients and suggest that determining the lowest dilution at which the sera has an effect on NMJ function could be a good strategy to evaluate the severity of the disease.

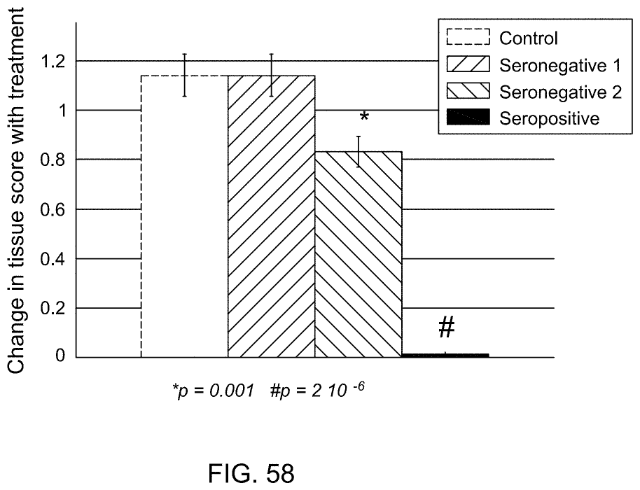

In order to prove the potential of the system as a diagnosis tool for myasthenia gravis in patients with undetectable levels of MG antibodies, the IgG fraction was isolated from 2 seronegative patients and 1 seropositive patient and tested. Result shows our system is able to detect a reduction in functionality in one of the seronegative samples (FIG. 58).

The system presented here is a human three-dimensional NMJ model that allows for automated quantification of function in a user-independent manner, which is accomplished through a unique combination of optogenetics, tissue engineering and image processing. Using this system, the ability to capture graded changes in NMJ function in response to physiologic and pathologic processes such as innervation, neurotoxin exposure, and myasthenia gravis is demonstrated.

The microfluidic device used in this work allowed for the controlled formation of functional NMJs between one human skeletal microtissue and one motoneuron neurosphere growing in separated compartments, allowing for the continued study over time of individual muscle-neurosphere pairs. Furthermore, the compartmentalized culture mimics human physiology more precisely than simpler coculture systems, and will allow for specific matrices and media for muscle formation, motoneuron maintenance, and axonal growth. Culture of three-dimensional muscle has been previously shown to better recapitulate the organization and function of native muscle. The presence of pillars also allows for the measurement of tissue forces by measuring their displacement. Direct comparison of electrical and optical evoked responses showed that while optical induced contractions are much smaller at early time points, they reached similar values to those evoked by electrical stimulation once the tissue was fully innervated.

The optical platform allowed for the controlled stimulation of the motoneurons using light pulses of controlled length and frequency. The results showed the system evoked single actions potentials that resulted in muscle contractions. The ramp protocol used in the experiments reported in this manuscript was 0.2 to 2 Hz. It was chosen to provide a good balance between the stimulation time, which directly correlates with the video size and processing time, and the distribution of results over the range of frequencies. This protocol is flexible and can be adapted to the experimental need or the tissue responsiveness.

Our video processing analysis is based on analyzing the response of the whole muscle tissue and not just muscle force. This eliminates the variability caused by small differences during tissue formation that can lead to different tissue geometries and therefore, forces. The software considers the existence of spontaneous contractions and takes them into account to generate the final score.

Using the system to track individual muscle-neurosphere pairs over time, the system was able to detect the gradual improvement of NMJ function during the first 2 weeks of coculture, followed by at least another week of stable function. These results provide insight into the NMJ formation process, and will allow for the screening of interventions that can lead to faster or more complete maturation.

Treatment with BTX, a neurotoxin that binds specifically to the acetylcholine receptors of the NMJ, completely stops muscle contractions, both spontaneous and light-induced. Without being bound to particular theory, this suggests that spontaneous contractions are due to spontaneous activity of the motoneurons and not the muscle and correlates with observations of non-innervated muscle tissues that did not show spontaneous activity.

Finally, the system modeled myasthenia gravis by incorporating patient sera in the NMJ model. The system showed very high sensitivity to the MG antibodies, and was able to clearly discriminate between samples from different patients. Furthermore, after removal of myasthenic antibodies, tissues showed functional recovery, mimicking the effect seen in MG patients when they undergo plasmapheresis. From a clinical standpoint, the ability to recapitulate the myasthenic phenotype not only the study of MG in a human in-vitro model of the NMJ, but also holds great potential as a diagnostic tool for MG and other pathologies such as LEMS. An optogenetic tissue-engineered human system disclosed herein could be used to evaluate MG and LEMS severity independently of the patient's serotype in a non-invasive way, and help with the differential diagnosis of these diseases in the context of NMJ disorders. In particular, a NMJ model disclosed herein should be able to distinguish between post-synaptic diseases such as MG, characterized by increased muscle weakness with repetition, and presynaptic disorders such as LEMS, which present with muscular improvement with repeated stimulation at high frequencies by using a ramp stimulation protocol. Furthermore, we demonstrated the capacity of our system to detect functional changes in at least a fraction of negative MG patients that test negative for any other existing test, proving the potential of this system to be used as a diagnostic tool for MG independently on the type of antibodies present.

The use of patient-derived stem cells allows for the study of genetic neuromuscular diseases in a mutation-specific manner, and the development of personalized medicine approaches for diagnosis and treatment.

While the disclosed subject matter is described herein in terms of certain non-limiting exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments. In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of non-limiting example embodiments of the disclosed subject matter has been presented for purposes of illustration and description. It is not intended to be exhaustive or to limit the disclosed subject matter to those embodiments disclosed herein. For example, improved spatial control of the light stimulation could be achieved by replacing LEDs by laser beams or by using micromirror technology to project specific illumination patterns. The concepts described herein may include introducing new cell types such as endothelial cells, Schwann cells, or spinal interneurons.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A system for evaluating the function of the neuromuscular junction (NMJ) of a subject, comprising
a platform including a body having a bottom, an open top, and first and second wells separated by a first raised lip having a first height, each well including
a first culture chamber;
a second culture chamber disposed adjacent to the first culture chamber, the first and second culture chambers separated by a second raised lip having a second height,
first and second pillars extending horizontally from a sidewall of the first culture chamber, and
a channel disposed at the bottom of the platform body extending between the first and second culture chambers;
the platform supporting a microtissue culture comprising:
human skeletal myoblasts in the first chamber, the human skeletal myoblasts derived from the subject, the first and second pillars providing a site for attachment of the myoblasts;
a neurosphere in the second chamber, the neurosphere derived from the subject, expressing an optogenetic protein, and
a hydrogel disposed in the channel to allow axonal growth between the myoblasts and the neurosphere;
a light source for optical stimulation pulses applied to the microtissue culture for activation of an optogenetic protein; and
an image recordation device for capturing images of the culture in response to the optical stimulation.

2. The system of claim 1, wherein the optogenetic protein is channelrhodopsin-2 (ChR2).

3. The system of claim 1, wherein the light source comprises a red 647 nm LED for brightfield illumination and a blue 488 nm LED for activation of the optogenetic protein.

4. The system of claim 1, wherein the light source comprises a controller to provide a ramp stimulation regimen comprising optical pulses delivered at successively higher frequencies.

5. The system of claim 4, wherein the pulses each comprise a duration of 100 milliseconds.

6. The system of claim 1, wherein the distance between the first and second pillars is 4 mm.

7. The system of claim 1, further comprising an image processor executing software configured to:
receive a stimulation trace of a plurality of optical stimulation pulses by the light source;
receive a series of image frames representative of NMJ motion in response to optical stimulation pulses by the light source;
extract motion by subtracting every image frame from a baseline frame;
create a trace of contractile activity comprising a plurality of contractions based on the subtraction;
align the trace of contractile activity against the stimulation trace; and
determine whether each of the optical stimulation pulses was effective based on the time period between an optical stimulation pulse and a contraction.

8. A tissue engineered three-dimensional model of the neuromuscular junction (NMJ) of a subject, comprising
a platform including a body having a bottom, an open top, and first and second wells separated by a first raised lip having a first height, each well including
a first culture chamber;
a second culture chamber disposed adjacent to the first culture chamber, the first and second culture chambers separated by a second raised lip having a second height,
first and second pillars extending horizontally from a sidewall of the first culture chamber, and
a channel disposed at the bottom of the platform body extending between the first and second culture chambers; the platform supporting a microtissue culture comprising:
human skeletal myoblasts disposed in the first chamber, the human skeletal myoblasts derived from the subject;
a neurosphere disposed in the second chamber, the neurosphere expressing an optogenetic protein, and
a hydrogel in the channel to allow axonal growth between the myoblasts and neurosphere.

9. The tissue-engineered three-dimensional model of claim 8, wherein the optogenetic protein is channelrhodopsin-2 (ChR2).

10. The tissue-engineered three-dimensional model of claim 8, wherein the human skeletal myoblasts comprise muscle-derived hiPSCs transduced with lentiviruses carrying an optogenetic protein.

11. The tissue engineered three-dimensional model of claim 8, wherein the microtissue defines a length of 4 mm.

12. A method of evaluating the function of the neuromuscular junction (NMJ) of a subject comprising:
providing a platform comprising first and second culture chambers separated by a gap portion; the platform supporting a culture comprising:
human skeletal myoblasts in the first chamber, the human skeletal myoblasts derived from the subject;
a neurosphere in the first chamber, the neurosphere derived from the subject, expressing an optogenetic protein,
a hydrogel in the gap portion to allow axonal growth between the myoblasts and neurosphere;
allowing axonal growth between the myoblasts and the neurosphere to form a tissue-engineered NMJ;
providing optical stimulation to the second chamber for activation of the optogenetic protein of the tissue-engineered NMJ;
measuring displacement of the tissue-engineered NMJ in response to the optical stimulation; and
evaluating the tissue culture by determining displacement of tissue in response to the optical stimulation.

13. The method of claim 12, wherein the optogenetic protein is channelrhodopsin-2 (ChR2).

14. The method of claim 12, wherein the evaluation comprises:
providing an image processor including software, the software when executed causes the image processor to
receive a stimulation trace of a plurality of optical stimulation pulses by the light source;
receive a series of image frames representative of NMJ motion in response to optical stimulation pulses by the light source;
extract motion by subtracting every image frame from a baseline frame;
create a trace of contractile activity comprising a plurality of contractions based on the subtraction;
align the trace of contractile activity against the stimulation trace; and
determine whether each of the optical stimulation pulses was effective based on the time period between an optical stimulation pulse and a contraction.

15. The method of claim 12, further comprising:
determining a ratio of effective pulses to total pulses.

16. The method of claim 12, further comprising:
exposing the tissue-engineered NMJ tissue to serum derived from a second subject; and
determining the presence of a neuromuscular disorder in the second subject based on a reduction in effective pulses following exposure of the NMJ tissue to the serum.

17. The method of claim 12, wherein providing optical stimulation comprises providing a red 647 nm LED for brightfield illumination and a blue 488 nm LED for activation of the ChR2.

18. The method of claim 12, wherein providing optical stimulation comprises providing a ramp stimulation regimen comprising pulses delivered at successively higher frequencies.

19. The method of claim 12, wherein providing optical stimulation comprises providing a plurality of pulses, each pulse having a duration of 100 milliseconds.

20. A bioreactor platform for evaluating the function of the neuromuscular junction (NMJ) of a subject, comprising
a body having a bottom, an open top, and first and second wells separated by a first raised lip having a first height, each well including
a first culture chamber;
a second culture chamber disposed adjacent to the first culture chamber, the first and second culture chambers separated by a second raised lip having a second height,
first and second pillars extending horizontally from a sidewall of the first culture chamber, and
a channel disposed at the bottom of the platform body extending between the first and second culture chambers.

21. The bioreactor platform of claim 20, wherein the first height is greater than the second height.

22. The bioreactor platform of claim 21, wherein the first culture chamber is disposed in a first section of the platform body, the first section including a bottom surface that is partially closed and an open top portion.

23. The bioreactor platform of claim 22, wherein a raised embossment surrounds the circumference of the first muscle chamber.

24. The bioreactor platform of claim 21, wherein the distance between the first and second pillars is 4 mm.
